(19) United States
(12) Patent Application Publication
(10) Pub. No.: US 2007/0009542 A1
(43) Pub. Date: Jan. 11, 2007

(54) Method and device for transdermal immunization

(76) Inventors: Galit Levin, Nordiya (IL); Amikam Gershonowitz, Modi'in (IL); Hana Gadasi, Mazkeret Batya (IL)

Correspondence Address: PEPPER HAMILTON LLP, ONE MELLON CENTER, 50TH FLOOR, 500 GRANT STREET, PITTSBURGH, PA, 15219, US

(21) Appl. No.: 11/324,007

(22) Filed: Dec. 30, 2005

Related U.S. Application Data
(63) Continuation-in-part of application No. PCT/IL05/00710, filed on Jul. 5, 2005.

(51) Int. Cl. A61N 1/30; A61K 39/00

(52) U.S. Cl. 424/184.1; 604/20

(57) ABSTRACT

The present invention relates to methods and device for transdermal immunization. More particularly, the invention relates to a device for effective topical administration of antigens comprising an apparatus that generates micro-channels in the skin of a subject. The delivery system is useful for immunization against bacterial, viral, and fungal antigens and for treating tumors and allergies.

### REFERENCE TO RELATED APPLICATIONS

This application is a continuation-in-part of PCT/IL2005/000710 entitled “Delivery System for Transdermal Immunization” filed on July ______, 2005, which is herein incorporated by reference in its entirety.

### BACKGROUND OF THE INVENTION

Vaccination can be achieved through various routes of administration, including oral, nasal, intramuscular (IM), subcutaneous (SC), and intradermal (ID). The majority of commercial vaccines are administered by IM or SC routes.

The skin's primary barrier, the stratum corneum, is impermeable to hydrophilic and high molecular weight drugs and macromolecules such as proteins, naked DNA, and viral vectors. Consequently, transdermal delivery has been generally limited to the passive delivery of low molecular weight compounds (<500 daltons) with limited hydrophilicity.

A number of approaches have been evaluated in an effort to circumvent the stratum corneum. Chemical permeation enhancers, depilatories and hydration techniques can increase skin permeability to macromolecules. However, these methods are relatively inefficient means of delivery. Furthermore, at nonirritating concentrations, the effects of chemical permeation enhancers are limited. Physical methods of permeation enhancement have also been evaluated, including sandpaper abrasion, tape stripping, and bifurcated needles. While these techniques increase permeability, it is difficult to predict the magnitude of their effect on drug absorption. Laser ablation may provide more reproducible effects, but it is currently cumbersome and expensive. Active methods of transdermal delivery include iontophoresis, electroporation, sonophoresis (ultrasound), and ballistic delivery of solid drug-containing particles. Delivery systems using active transport (e.g., sonophoresis) are in development, and delivery of macromolecules is possible with such systems. However, at this stage, it is not yet known if these systems will allow successful and reproducible delivery of macromolecules in humans.

Pathogens entering the skin are confronted with a highly organized and diverse population of specialized cells capable of eliminating microorganisms through a variety of mechanisms. Epidermal Langerhans cells are potent antigen-presenting cells. Lymphocytes and dermal macrophages can penetrate to the dermis. Keratinocytes and Langerhans cells express or can be induced to generate a diverse array of immunologically active compounds. Collectively, these cells orchestrate a complex series of events that ultimately control both innate and specific immune responses.

PCT International Patent Applications WO 2004/039426; WO 2004/039427; and WO 2004/039428, all assigned to the applicant of the present application, disclose systems and methods for transdermal delivery of pharmaceutical agents. Specifically disclosed are hydrophilic anti-emetic agents, dried compositions comprising polypeptides and proteins, and water-insoluble drugs. The systems and methods disclosed in WO 2004/039426, WO 2004/039427, and WO 2004/039428 significantly increased the permeation of the pharmaceutical compositions to the blood.

### SUMMARY OF THE INVENTION

One embodiment of the present invention relates to a transdermal delivery system for immunization. The transdermal delivery system comprises an apparatus that generates a plurality of micro-channels in an area of the skin of a subject and a composition comprising an antigenic agent.

In one embodiment, the transdermal delivery system of the present invention does not require an adjuvant. The immunizing effect achieved by the system of the present invention can be seen in the absence of an adjuvant as well as in its presence, and thus rescues the skin area to which the antigenic agent is applied from irritation, sensitization or toxic effects associated with the use of an adjuvant. A composition comprising an antigenic agent or a commercially available vaccine can be administered in conjunction with the apparatus of the present invention, as it is shown herein that the micro-channels generated by the apparatus of the present invention enable effective delivery of a vaccine into the subject's body and induction of an antigen-specific immune response.

It is further disclosed that the delivery systems of the present invention is highly useful for inducing an immune response against high molecular weight molecules. The immune response induced is not limited to one antibody subtype, but rather can include the production of several antibody subtypes, i.e., IgM, IgG, and IgA.

It is further disclosed that treatment of an area of the skin of a subject with the apparatus of the present invention and subsequent topical application of an antigenic agent on the area of the skin of the subject, increases the IgA and the IgG antibody titers specific to the antigenic agent and these titers are comparable or even higher than those obtained by conventional immunization routes, i.e., subcutaneous or intramuscular routes. Thus, the present invention provides a system for immunization or vaccination that avoids the need for injections.

Treatment of an area of the skin of a subject with the apparatus of the present invention and then topical application of an antigenic agent on the area of the skin of the subject results in earlier appearance of significant and detectable titers of IgG antibodies specific to the antigenic agent as compared to the time of appearance of antibodies subsequent to subcutaneous or intramuscular antigen administration. Thus, for many applications, which require a rapid onset of immunity, the system of the present invention is specifically advantageous.

It is further disclosed that topical application of a solution comprising an antigenic agent on an area of the skin of a subject, which has been treated with the apparatus of the present invention, elicits antigen specific IgG antibodies more efficiently than a patch comprising a dried antigenic agent that is applied on skin treated with said apparatus. However, treatment of skin with the apparatus of the present invention and then application of a patch comprising a dried antigenic agent on the treated skin is shown to be highly efficient in eliciting antigen specific IgA antibodies as compared to subcutaneous or intramuscular routes. Thus, the apparatus of the present invention in conjunction with a particular formulation of an antigenic agent is useful for manipulating the immune system.

It is explicitly intended that the present invention encompasses a wide variety of bacterial antigens, viral antigens, fungal antigens and other high molecular weight agents capable of inducing an antigen-specific immune response. The principles of the present invention are exemplified herein below using ovalbumin, a 45 kDa protein, and inactivated influenza vaccine consisting of three strains originally isolated from humans.

According to one embodiment, the present invention provides a transdermal delivery system for inducing an antigen-specific immune response comprising an apparatus for facilitating transdermal delivery of an antigen through an area of the skin of a subject, wherein the apparatus capable of generating a plurality of micro-channels in the area of the skin of the subject other than by mechanical means, and a composition comprising an immunogenically effective amount of an antigen.

According to some embodiments, the present invention incorporates the techniques for creating micro-channels by inducing ablation of the stratum corneum by electrical energy including the devices disclosed in U.S. Pat. Nos. 6,148,232; 6,597,946; 6,611,706; 6,711,435; and 6,708,060; the contents of which are incorporated by reference as if fully set forth herein. It is, however, emphasized that although some preferred embodiments of the present invention relate to intradermal or transdermal antigen delivery obtained by ablating the skin by the aforementioned apparatus, substantially any method known in the art for generating micro-channels in the skin of a subject can be used, except of methods utilizing mechanical means.

In a further embodiment, the transdermal delivery system comprising the apparatus for facilitating transdermal delivery of an antigen through an area of the skin of a subject, said apparatus comprises:

an electrode cartridge comprising a plurality of electrodes; and

a main unit comprising a control unit which is adapted to apply electrical energy to the plurality of electrodes when said plurality of electrodes are in vicinity of the skin, typically generating current flow or one or more sparks, enabling ablation of stratum corneum in an area beneath the electrodes, thereby generating the plurality of micro-channels.

According to another embodiment, the control unit of the apparatus comprises circuitry to control the magnitude, frequency, and/or duration of the electrical energy delivered to the electrodes, so as to control the current flow or spark generation, and thus the width, depth and shape of the plurality of micro-channels. This provides, heretofore, a mechanism to provide channels of width, depth and shape suitable for vaccine delivery. The electrical energy may be, for example, at radio frequency.

According to one embodiment, the electrode cartridge comprising the plurality of electrodes generates a plurality of micro-channels having uniform shape and dimensions. According to some embodiments, the electrode cartridge is removable. The electrode cartridge can be discarded after one use, and as such it is designed for easy attachment to the main unit and subsequent detachment from the main unit.

According to some embodiments, the antigen is selected from bacterial antigens, viral antigens, fungal antigens, protozoan antigens, tumor antigens, allergens, autoantigens, fragments, analogs, derivatives thereof, and combinations thereof.

According to additional embodiments, the bacterial antigen is derived from a bacterium selected from anthrax, Campylobacter, Vibrio cholera, clostridia, Diphtheria, enterohemorrhagic E coli, enterotoxigenic E. coli, Giardia, gonococcus, Helicobacter pylori, Hemophilus influenza B, Hemophilus influenza non-typeable, Legionella, meningococcus, Mycobacteria, pertussis, pneumococcus, salmonella, shigella, staphylococcus, Group A beta-hemolytic streptococcus, Streptococcus B, tetanus, Borrelia burgdorfi, Yersinia and combinations thereof.

According to other embodiments, the viral antigen is derived from a virus selected from adenovirus, ebola virus, enterovirus, hanta virus, hepatitis virus, herpes simplex virus, human immunodeficiency virus, human papilloma virus, influenza virus, measles (rubeola) virus, Japanese equine encephalitis virus, papilloma virus, parvovirus B19, poliovirus, respiratory syncytial virus, rotavirus, St. Louis encephalitis virus, vaccinia virus, yellow fever virus, rubella virus, chickenpox virus, varicella virus, mumps virus and combinations thereof.

According to other embodiments, the fungal antigen is derived from a fungus selected from tinea corporis, tinea unguis, sporotrichosis, aspergillosis, candida, and combinations thereof.

According to additional embodiments, the protozoan antigen is derived from protozoa selected from Entamoeba histolytica, Plasmodium, Leishmania and combinations thereof.

According to some embodiments, the antigen is selected from peptides, polypeptides, proteins, glycoproteins, lipoproteins, lipids, phospholipids, carbohydrates, glycolipids, conjugates thereof and combinations thereof. It is to be understood that the composition can comprise two or more antigens.

According to yet other embodiments, the composition comprising the antigen of the invention can be formulated in a dry formulation or liquid formulation. According to an exemplary embodiment, the dry formulation is a patch.

According to some embodiments, the composition comprising the antigen further comprises an adjuvant.

According to another aspect, the present invention provides a method for inducing transdermally an antigen-specific immune response in a subject comprising:

generating a plurality of micro-channels in an area of the skin of a subject other than by mechanical means; and

topically applying a composition comprising an immunogenically effective amount of an antigen and a pharmaceutically acceptable carrier to the area of the skin in which the plurality of micro-channels are present, thereby inducing an antigen-specific immune response.

According to some embodiments, the plurality of micro-channels are generated by an apparatus comprising:

an electrode cartridge comprising a plurality of electrodes; and

a main unit comprising a control unit which is adapted to apply electrical energy to the plurality of electrodes when said plurality of electrodes are in vicinity of the skin, typically generating current flow or one or more sparks, enabling ablation of stratum corneum in an area beneath the electrodes, thereby generating the plurality of micro-channels.

According to additional embodiments, the electrode cartridge comprising the plurality of electrodes is removable. According to further embodiments, the electrical energy may be, for example, radio frequency.

According to some embodiments, the method for inducing an antigen-specific immune response comprises an antigen-specific antibody. According to additional embodiments, the antigen-specific immune response comprises an antigen-specific lymphocyte.

It is to be understood that as the method for transdermally inducing an immune response according to the principles of the present invention enables eliciting the response against a variety of antigenic agents such as bacterial antigens, viral antigens, fungal antigens, protozoan antigens, tumor antigens, allergens, and autoantigens, the method of the present invention is useful for immunoprotection, immunosuppression, modulation of an autoimmune disease, potentiation of cancer immunosurveillance, prophylactic vaccination to prevent disease, and therapeutic vaccination to treat or reduce the severity and/or duration of established disease.

These and other embodiments of the present invention will be better understood in relation to the figures, description, examples and claims that follow.

### BRIEF DESCRIPTION OF THE FIGURES

For a fuller understanding of the nature and advantages of the present invention, reference should be made to the following detailed description taken in connection with the accompanying drawings, in which:

FIG. 1 shows IgM plasma titers in guinea pigs 15 days after either primary subcutaneous immunization (S.C.) with ovalbumin or ViaDerm treatment followed by transdermal immunization with ovalbumin solution (VD-s).

FIG. 2 shows IgG plasma titers in guinea pigs 15 days after either primary subcutaneous immunization (S.C.) with ovalbumin or ViaDerm treatment followed by transdermal immunization with ovalbumin solution (VD-s).

FIGS. 3A-B show IgA and IgG plasma titers in guinea pigs 6 days after boost (day 36 after primary immunization). FIG. 3A shows IgA and IgG plasma titers 6 days after boost (day 36 after primary immunization) by intramuscular immunization with ovalbumin solution (i.m.) or subcutaneous immunization (S.C.) with ovalbumin. FIG. 3B shows IgA and IgG plasma titers 6 days after boost (day 36) by ViaDerm treatment followed by transdermal immunization with either ovalbumin solution (VD-s) or ovalbumin powder (VD-p).

FIG. 4 shows IgG plasma titers in guinea pigs 95 days after boost (125 days after primary vaccination) by either subcutaneous immunization (S.C.) with ovalbumin or ViaDerm treatment followed by transdermal immunization with ovalbumin solution (VD-s).

FIG. 5 shows IgA plasma titers in guinea pigs 15 days after either primary subcutaneous immunization (S.C.) with ovalbumin or ViaDerm treatment followed by transdermal immunization with ovalbumin solution (VD-s).

FIG. 6 shows IgA plasma titers in guinea pigs 12 days after boost (day 42 after primary immunization) by either subcutaneous immunization (S.C.) with ovalbumin or ViaDerm treatment followed by transdermal immunization with ovalbumin solution (VD-s).

FIG. 7 shows Trans Epidermal Water Loss (TEWL) values in guinea pigs treated with either 50-micron or 100-micron length electrodes of ViaDerm and control guinea pigs.

FIG. 8 shows serum IgG antibody titers against A/Panama strain of influenza in guinea pigs treated with either 50-micron or 100-micron length electrodes of ViaDerm and then immunized with the influenza vaccine patch in the absence or presence of the adjuvant E. coli heat labile enterotoxin (LT). A control group was immunized with the influenza vaccine patch in the absence or presence of LT. A group of guinea pigs immunized intramuscularly with the influenza vaccine and then boosted intramuscularly with the same vaccine is also shown.

FIG. 9 shows serum IgG antibody titers against A/Caledonia strain of influenza in guinea pigs treated with either 50-micron or 100-micron length electrodes of ViaDerm and then immunized with the influenza vaccine patch in the absence or presence of LT. A control group was immunized with the influenza vaccine patch in the absence or presence of LT. A group of guinea pigs immunized intramuscularly with the influenza vaccine and then boosted intramuscularly with the same vaccine is also shown.

FIG. 10 shows serum IgG antibody titers against B/Shangdong strain of influenza in guinea pigs treated with either 50-micron or 100-micron length electrodes of ViaDerm and then immunized with the influenza vaccine patch in the absence or presence of LT. A control group was immunized with the influenza vaccine patch in the absence or presence of LT. A group of guinea pigs immunized intramuscularly with the influenza vaccine and then boosted intramuscularly with the same vaccine is also shown.

FIG. 11 shows serum IgG antibody titers against the A/Wyoming strain of influenza in guinea pigs treated with 50-micron length electrodes of ViaDerm and then immunized with a influenza vaccine patch in the absence or presence of LT. A group was immunized with the influenza vaccine patch in the absence or presence of LT after mechanical application of an ECG patch. A group of guinea pigs immunized intramuscularly with the influenza vaccine and then boosted intramuscularly with the same vaccine is also shown.

FIG. 12 shows serum IgG antibody titers against the A/Caledonia strain of influenza in guinea pigs treated with 50-micron length electrodes of ViaDerm and then immunized with a influenza vaccine patch in the absence or presence of LT. A group was immunized with the influenza vaccine patch in the absence or presence of LT after mechanical application of an ECG patch. A group of guinea pigs immunized intramuscularly with the influenza vaccine and then boosted intramuscularly with the same vaccine is also shown.

FIG. 13 shows serum IgG antibody titers against the B/Jiangsu strain of influenza in guinea pigs treated with 50-micron length electrodes of ViaDerm and then immunized with a influenza vaccine patch in the absence or presence of LT. A group was immunized with the influenza vaccine patch in the absence or presence of LT after mechanical application of an ECG patch. A group of guinea pigs immunized intramuscularly with the influenza vaccine and then boosted intramuscularly with the same vaccine is also shown.

FIG. 14 shows serum IgG antibody titers against A/Wyoming, A/Caledonia and B/Jiangsu strains of influenza in guinea pigs treated with 100-micron length electrodes of ViaDerm and then immunized with a liquid or dry influenza vaccine patch in the absence or presence of LT. A group was immunized with the influenza vaccine patch in the absence or presence of LT with hydration. A group was immunized with the influenza vaccine patch in the absence or presence of LT with hydration. A group of guinea pigs immunized intramuscularly with the influenza vaccine and then boosted intramuscularly with the same vaccine is also shown.

FIG. 15 shows serum IgG antibody titers against A/Wyoming, A/Caledonia and B/Jiangsu strains of influenza in hairy guinea pigs treated with 100-micron length electrodes of ViaDerm and then immunized with a liquid or dry influenza vaccine patch in the absence or presence of LT. A group of guinea pigs immunized intramuscularly with the influenza vaccine and then boosted intramuscularly with the same vaccine is also shown.

FIG. 16 shows serum IgG antibody titers against A/Wyoming, A/Caledonia and B/Jiangsu strains of influenza in shaved guinea pigs treated with ECG buffer pad and then immunized with a dry influenza vaccine patch in the presence of BLT or iLT or no adjuvant.

FIG. 17 shows serum IgG antibody titers against A/Wyoming, A/Caledonia and B/Jiangsu strains of influenza in shaved guinea pigs treated with 50-micron length electrodes of ViaDerm and then immunized with a dry influenza vaccine patch in the presence of BLT or iLT or no adjuvant.

### DETAILED DESCRIPTION OF THE INVENTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

One embodiment of the present invention provides transdermal delivery system for inducing an antigen-specific immune response comprising an apparatus for facilitating transdermal delivery of an antigenic agent through the skin of a subject, said apparatus capable of generating at least one micro-channel in an area on the skin of the subject and a composition comprising an immunogenically effective amount of at least one antigenic agent.

As used herein, the term “about” means plus or minus 10% of the numerical value of the number with which it is being used. Therefore, about 50% means in the range of 45%-55%.

Antigen.

The terms “antigenic agent” and “antigen”, used interchangeably throughout the specification and claims, refer to an active component of the composition, which is specifically recognized by the immune system of a human or animal subject after immunization or vaccination. The antigen can comprise a single or multiple immunogenic epitopes recognized by a B-cell receptor (i.e., secreted or membrane-bound antibody) or a T cell receptor.

The antigenic agent according to the present invention is also an immunogenic agent. An “immunogenic” agent refers to an agent that is capable of inducing an antigen specific immune response.

The terms “immunization” and “vaccination” refer to the induction of an antigen specific immune response and are used interchangeably throughout the specification and claims.

An antigen can be a peptide, a polypeptide, a protein, a glycoprotein, a lipoprotein, a lipid, a phospholipid, a carbohydrate, a glycolipid, a mixture or a conjugate thereof, or any other material known to induce an immune response. The molecular weight of the antigen may be greater than 1 kilodalton (kDa), 10 kDa or 100 kDa (including intermediate ranges thereof). An antigen can be conjugated to a carrier. An antigen can be provided as a whole organism such as, for example, a bacterium or virion; an antigen can be obtained from an extract or lysate of organisms, e.g., from whole cells or from membranes; an antigen can be provided as live organisms such as, for example, live viruses or bacteria, attenuated live organisms such as, for example, attenuated live viruses or bacteria, or organisms that have been inactivated by chemical or genetic techniques; and an antigen can be chemically synthesized, produced by recombinant technology or purified from natural sources.

A “peptide” refers to a polymer in which the monomers are amino acids linked together through amide bonds. Peptides are generally smaller than polypeptides, typically under 30-50 amino acids in total.

A “polypeptide” refers to a single polymer of amino acids, generally over 50 amino acids.

A “protein” as used herein refers to a polymer of amino acids typically over 50 amino acids comprising one or more polypeptide chains.

Antigenic peptides or polypeptides include, for example, natural, synthetic or recombinant B-cell or T-cell epitopes, universal T-cell epitopes, and mixed T-cell epitopes from one organism or disease and B-cell epitopes from another. Antigens obtained through recombinant technology or peptide synthesis as well as antigens obtained from natural sources or extracts can be purified by purification methods based on the physical and chemical characteristics of the antigens, preferably by fractionation or chromatography. Peptide synthesis is well known in the art and is available commercially from a variety of companies. A peptide or polypeptide can be synthesized using standard direct peptide synthesis (e.g., as summarized in Bodanszky, 1984, Principles of Peptide Synthesis (Springer-Verlag, Heidelberg), such as via solid-phase synthesis (see, e.g., Merrifield, 1963, J. Am. Chem. Soc. 85:2149-2154).

Recombinant antigens can combine one or more antigens. An antigen composition comprising one or more antigens can be used to induce an immune response to more than one antigen at the same time. Such recombinant antigens can be made by ligating the appropriate nucleic acid sequences encoding the desired amino acid sequences to each other by methods known in the art, in the proper coding frame, and expressing the recombinant antigens by methods commonly known in the art (see, for example, Sambrook et al., 1989, Molecular Cloning: A Laboratory Manual, 2d edition, Cold Spring Harbor Press). Additionally or alternatively, a multivalent antigen composition can be used to induce an immune response to more than one immunogenic epitope in one antigenic agent. Conjugates can also be used to induce an immune response to multiple antigens, to boost the immune response, or both. Such conjugates can be made by protein synthesis, e.g., by use of a peptide synthesizer. Fragments of antigens can be also used to induce an immune response.

Many antigens can be used to vaccinate a subject and to induce an immune response specific for the antigen. The antigen can be derived from a pathogen that can infect a subject. Thus, antigens can be derived from, for example, bacteria, viruses, fungi, or parasites. The antigen can be a tumor antigen. The antigen can be an allergen including, but not limited to, pollen, animal dander, mold, dust mite, flea allergen, salivary allergen, grass, or food (e.g., peanuts and other nuts). The antigen can be an autoantigen. The autoantigen can be associated with an autoimmune disease such as, for example, the pancreatic islet antigen.

Antigens can be derived from bacteria. Examples of bacteria include, but are not limited to, anthrax, Campylobacter, Vibrio cholera, clostridia including Clostridium difficile, Diphtheria, enterohemorrhagic E. coli, enterotoxgenic E. coli, Giardia, gonococcus, Helicobacter pylori, Hemophilus influenza B, Hemophilus influenza non-typeable, Legionella, meningococcus, Mycobacteria including those organisms responsible for tuberculosis, pertussis, pneumococcus, salmonella, shigella, staphylococcus, Group A beta-hemolytic streptococcus, Streptococcus B, tetanus, Borrelia burgdorfi, Yersinia, and the like. According to the present invention, bacterial antigens include, for example, toxins, toxoids (i.e., chemically inactivated toxins, which are less toxic but retain immunogenicity), subunits or combinations thereof, and virulence or colonization factors. Bacterial constituents, products, lysates and/or extracts can be used as a source for bacterial antigens.

Antigens can be derived from viruses. Viruses include, but are not limited to, adenovirus, dengue serotypes 1 to 4 virus, ebola virus, enterovirus, hanta virus, hepatitis virus serotypes A to E, herpes simplex virus 1 or 2, human immunodeficiency virus, human papilloma virus, influenza virus, measles (rubeola) virus, Japanese equine encephalitis virus, papilloma virus, parvovirus B19, poliovirus, rabies virus, respiratory syncytial virus, rotavirus, St. Louis encephalitis virus, vaccinia virus, yellow fever virus, rubella virus, chickenpox virus, varicella virus, and mumps virus. Viral constituents, products, lysates and/or extracts can be used as a source for the viral antigens.

Antigens can be derived from fungi. Fungi include, but are not limited to, tinea corporis, tinea unguis, sporotrichosis, aspergillosis, candida, and other pathogenic fungi. Fungal constituents, products, lysates and/or extracts can be used as a source for the fungal antigens.

Antigens can be produced from protozoans. Protozoans include, for example, Entamoeba histolytica, Plasmodium, and Leishmania. Protozoan constituents, products, lysates and/or extracts can be used as a source for the protozoan antigens.

Vaccination can be also used as a treatment for cancer, allergies, and autoimmune diseases. For example, vaccination with a tumor antigen (e.g., HER2, prostate specific antigen) can induce an immune response in the form of antibodies and lymphocyte proliferation, which allows the body's immune system to recognize and kill tumor cells. Tumor antigens useful for vaccination are known in the art and include, for example, tumor antigens of leukemia, lymphoma, and melanoma.

Vaccination with T-cell receptors or autoantigens (e.g., pancreatic islet antigen) can induce an immune response that halts progression of an autoimmune disease.

It is to be understood that the present invention encompasses fragments, derivatives, and analogs of the antigenic agents so long as the fragments, derivatives, and analogs being immunogenic and thereby capable of inducing an antigen specific immune response.

Fragments of an antigenic agent can be produced by subjecting the antigen to at least one cleavage agent. A cleavage agent can be a chemical cleavage agent, e.g., cyanogen bromide, or an enzyme, e.g., endoproteinase, exoproteinase, or lipase.

Derivatives of the antigenic agents are also included in the scope of the present invention. Thus, protein antigenic agents can be modified by derivatization reactions including, but not limited to, oxidation, reduction, myristylation, sulfation, acylation, ADP-ribosylation, amidation, cyclization, disulfide bond formation, hydroxylation, iodination, methylation, glycosylation, deglycosylation, phosphorylation, dephosphorylation or any other derivatization method known in the art. Such alterations, which do not destroy the immunogenic epitope of an antigen, can occur anywhere in the antigen. It will be appreciated that one or more modifications can be present in the same antigen.

The term “analog” as used herein refers to antigenic agents comprising altered sequences by amino acid substitutions, additions or deletions.

Adjuvant.

The present invention provides highly effective systems and methods for transdermal delivery of antigenic agents without the use of adjuvants. However, embodiments of the present invention may also encompasses compositions comprising an antigen and an adjuvant. Generally, activation of antigen presenting cells by an adjuvant occurs prior to presentation of an antigen. Alternatively, an antigen and an adjuvant can be separately presented within a short interval of time but targeting the same anatomical region.

The term “adjuvant” refers to a substance that is used to specifically or nonspecifically potentiate an antigen-specific immune response. The term “adjuvant activity” is the ability to increase the immune response to an antigen (i.e., an antigen which is a separate chemical structure from the adjuvant) by inclusion of the adjuvant in a composition or as part of a method.

Adjuvants include, but are not limited to, an oil emulsion (e.g., complete or incomplete Freud's adjuvant), chemokines (e.g., defensins, HCC-1, HCC-4, MCP-1, MCP-3, MCP-4, MIP-1α, MIP-1β, MIP-1δ, MIP-3α, and MIP-2); other ligands of chemokine receptors (e.g., CCR-1, CCR-2, CCR-5, CCR-6, CXCR-1); cytokines (e.g., IL-1, IL-2, IL-6, IL-8, IL-10, IL-12, IFN-γ; TNF-α, GM-CSF); other ligands of receptors for these cytokines, immunostimulatory CpG motifs of bacterial DNA or oligonucleotides; muramyl dipeptide (MDP) and derivatives thereof (e.g., murabutide, threonyl-MDP, muramyl tripeptide); heat shock proteins and derivatives thereof; Leishmania homologs and derivatives thereof, bacterial ADP-ribosylating exotoxins, chemical conjugates and derivatives thereof (e.g., genetic mutants, A and/or B subunit-containing fragments, chemically toxoid versions); or salts (e.g., aluminum hydroxide or phosphate, calcium phosphate).

Most ADP-ribosylating exotoxins (bARE) are organized as A:B heterodimers with a B subunit containing the receptor binding activity and an A subunit containing the ADP-ribosyltransferase activity. Exemplary bARE include cholera toxin (CT), E. coli heat-labile enterotoxin (LT), diphtheria toxin, Pseudomonas exotoxin A (ETA), pertussis toxin (PT), C. botulinum toxin C2, C. botulinum toxin C3, C. limosum exoenzyme, B. cereus exoenzyme, Pseudomonas exotoxin S, S. aureus EDIN, and B. sphaericus toxin. Mutant bARE containing mutations of the trypsin cleavage site or mutations affecting ADP-ribosylation may be used.

It is to be understood that adjuvants such as bARE are known to be highly toxic when injected or given systemically. But if placed on the surface of intact skin or penetrate to the epidermis, they can provide adjuvant effects without systemic toxicity (see, for example, U.S. Patent Application Publication Nos. 2004/0258703 and 2004/0185055, incorporated by reference as if fully set for the herein).

Adjuvant can be chosen to preferentially induce specific antibodies (e.g., IgM, IgD, IgA1, IgA2, IgE, IgG1, IgG2, IgG3, and/or IgG4), or specific T-cell subsets (e.g., CTL, Th1, and/or Th2).

Unmethylated CpG dinucleotides or similar motifs are known to activate B lymphocytes and macrophages. Other forms of bacterial DNA can be used as adjuvants. It is to be understood that bacterial DNA belongs to a class of structures, which have patterns allowing the immune system to recognize their pathogenic origin and to stimulate the innate immune response leading to adaptive immune responses. These structures are called pathogen-associated molecular patterns (PAMP) and include lipopolysaccharides, teichoic acids, unmethylated CpG motifs, double stranded RNA, and mannins. PAMP induce endogenous signals that can mediate the inflammatory response and can act as co-stimulators of T-cell function.

Adjuvants can be biochemically purified from a natural source, can be produced synthetically or recombinantly produced. The adjuvants according to the present invention include truncations, substitutions, deletions, and additions of the natural occurring adjuvants so long as the adjuvant activity is retained.

Compositions.

Currently, licensed vaccines are delivered in an aqueous solution or suspension, and administered by the intramuscular or oral route during immunization. The drawbacks of mixing vaccine components with water or buffers under conditions of questionable sterility and the possibility that antigens in solution will break down are well known and, in part, has led to the need for cold storage of vaccine components. Vaccine components in the presence of water are chemically less stable and more prone to contamination through the provision of an aqueous medium for the growth of bacteria. The stringent requirement for cold storage during transport and storage of vaccines has led to the ‘cold chain’, indicating that at all times after manufacture of the vaccine, the vaccine is kept in proper cold storage conditions. This increases the complexity of storing vaccine, creates logistical problems when transporting vaccine, and adds greatly to the expense of vaccination.

The compositions useful for immunization or vaccination according to the present invention contain an immunogenically effective amount of at least one antigenic agent and a pharmaceutically acceptable carrier or vehicle in order to provide pharmaceutical-acceptable compositions suitable for administration to a subject (i.e., human or animal).

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic compound is administered. Thus, according to the invention, antigens can be solubilized in a buffer or water, or incorporated in emulsions, lipid micelles or vesicles. Suitable buffers include, but are not limited to, phosphate buffered saline (PBS), phosphate buffered saline Ca++/Mg++ free, normal saline (150 mM NaCl in water), Hepes or Tris buffer. Antigens, which are not soluble in neutral buffer, can be solubilized in 10 mM acetic acid and then diluted to the desired volume with a neutral buffer such as PBS. In the case of an antigen, which is soluble only at acidic pH, acetate-PBS at acidic pH can be used as a diluent after solubilization in dilute acetic acid. Other useful carriers include, for example, ethanol, ethylene glycol, propylene glycol, butane-1,3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990.

Optionally, components like stabilizers, colorings, humectants, preservatives, adhesives, plasticizers, tackifiers, and thickeners can be included in the composition.

Stabilizers include, but are not limited to, dextrans and dextrins, glycols, alkylene glycols, polyalkane glycols, polyalkylene glycols, sugars, starches, and derivatives thereof. Preferred additives are non-reducing sugars and polyols. In particular, glycerol, trehalose, hydroxymethyl or hydroxyethyl cellulose, ethylene or propylene glycol, trimethyl glycol, vinyl pyrrolidone, and polymers thereof can be added. Alkali metal salts, ammonium sulfate, and magnesium chloride can stabilize proteinaceous antigens. A polypeptide can also be stabilized by contacting it with a sugar such as, for example, a monosaccharide, disaccharide, sugar alcohol, and mixtures thereof (e.g., arabinose, fructose, galactose, glucose, lactose, maltose, mannitol, mannose, sorbitol, sucrose, xylitol). Polyols can also stabilize a polypeptide. Various other excipients can also stabilize polypeptides including amino acids, phospholipids, reducing agents, and metal cheating agents.

The compositions of the invention can be formulated as a dry or liquid formulation. A dry formulation is more easily stored and transported than conventional liquid vaccines, as it breaks the cold chain required from the vaccine's place of manufacturing to the location where vaccination occurs. In addition, a dry formulation can be more advantageous than liquid formulations since high concentrations of a dry active component of the composition (e.g., one or more antigens) can be achieved by solubilization directly at the site of immunization over a short time span. Moisture from the skin and an occlusive backing layer can hasten this process.

The composition can be provided as a liquid formulation including, but not limited to, solution, suspension, emulsion, cream, gel, lotion, ointment, paste, or other liquid forms.

The composition can be provided as a dry formulation. Dry formulations include, but not limited to, fine or granulated powders, uniform films, pellets, tablets and patches. The formulation may be dissolved and then dried in a container or on a flat surface (e.g., skin), or it may simply be dusted on the flat surface. It may be air dried, dried with elevated temperature, freeze or spray dried, coated or sprayed on a solid substrate and then dried, dusted on a solid substrate, quickly frozen and then slowly dried under vacuum, or combinations thereof. If more than one antigenic agent is included in a composition, the antigenic agents can be mixed in solution and then dried, or mixed in a dry form only.

The composition can be provided in a form of a patch. A “patch” refers to a product, which comprises an antigenic agent and a solid substrate, typically a backing layer, which functions as the primary structural element of the patch (see, for example, WO 02/074244 and WO 2004/039428, incorporated by reference as if fully set forth herein). A patch can further comprise an adhesive and/or a microporous liner layer. Typically, the microporous liner layer is a rate-controlling matrix or a rate-controlling membrane that allow extended release of the antigenic agent.

A liquid formulation can be incorporated in a patch (i.e., a wet patch). The liquid formulation can be held in a reservoir or can be mixed with the contents of a reservoir. A wet patch can contain a single reservoir containing one antigenic agent, or multiple reservoirs to separate individual antigenic agents.

A patch can also be a dry patch. A dry patch can be a powder patch such as, for example, a printed patch as disclosed in WO 2004/039428 or any other dry patch known in the art; applying a powder patch allows control over the time and rate of the dissolution of the antigenic agent. A dry patch can include one or more dried antigenic agents such that application of a patch, whether a wet or dry patch, comprising multiple antigens induces an immune response to the multiple antigens. In such a case, antigens can or cannot be derived from the same source, but will have different chemical structures so as to induce an immune response specific for the different antigens.

The backing layer can be non-woven or woven (e.g., gauze dressing). It may be non-occlusive or occlusive, but the latter is preferred. The optional release liner preferably does not adsorb significant amounts of the composition. The patch is preferably hermetically sealed for storage (e.g., foil packaging). The patch can be held onto the skin and components of the patch can be held together using various adhesives. One or more of the antigens may be incorporated into the substrate or adhesive parts of the patch. Generally, patches are planar and pliable, and they are manufactured with a uniform shape. Optional additives are plasticizers to maintain pliability of the patch, tackifiers to assist in adhesion between patch and skin, and thickeners to increase the viscosity of the formulation at least during processing.

Metal foil, cellulose, cloth (e.g., acetate, cotton, rayon), acrylic polymer, ethylenevinyl acetate copolymer, polyamide (e.g., nylon), polyester (e.g., polyethylene naphthalate, ethylene terephthalate), polyolefin (e.g., polyethylene, polypropylene), polyurethane, polyvinyl alcohol, polyvinyl pyrrolidone, polyvinylidene chloride (SARAN), natural or synthetic rubber, silicone elastomer, and combinations thereof are examples of patch materials (e.g., backing layer, release liner).

The adhesive may be an aqueous-based adhesive (e.g., acrylate or silicone). Acrylic adhesives, available from several commercial sources, are sold under the trade names AROSET, DUROTAK, EUDRAGIT, GELVA, and NEOCRYL.

For the purpose of increasing or decreasing the water absorption capacity of an adhesive layer, the acrylic polymer may be co-polymerized with hydrophilic monomer, monomer containing carboxyl group, monomer containing amide group, monomer containing amino group, and the like. Rubbery or silicone resins may be employed as the adhesive resin; they may be incorporated into the adhesive layer with a tackifying agent or other additives.

Alternatively, the water absorption capacity of the adhesive layer can be also regulated by incorporating therein highly water-absorptive polymers, polyols, and water-absorptive inorganic materials. Examples of the highly water-absorptive resins may include mucopolysaccharides such as hyaluronic acid, chondroitin sulfate, dermatan sulfate and the like; polymers having a large number of hydrophilic groups in the molecule such as chitin, chitin derivatives, starch and carboxy-methylcellulose; and highly water-absorptive polymers such as polyacrylic, polyoxyethylene, polyvinyl alcohol, and polyacrylonitrile.

The plasticizer may be a trialkyl citrate such as, for example, acetyl-tributyl citrate (ATBC), acetyl-triethyl citrate (ATEC), and triethyl citrate (TEC). Exemplary tackifiers are glycols (e.g., glycerol, 1,3 butanediol, propylene glycol, polyethylene glycol). Succinic acid is another tackifier.

Thickeners can be added to increase the viscosity of an adhesive or immunogenic composition. The thickener may be a hydroxyalkyl cellulose or starch, or water-soluble polymers: for example, poloxamers, polyethylene oxides and derivatives thereof, polyethyleneimines, polyethylene glycols, and polyethylene glycol esters. However, any molecule which serves to increase the viscosity of a solution may be suitable to improve handling of a formulation during manufacture of a patch.

Gel and emulsion systems can be incorporated into patch delivery systems, or be manufactured separately from the patch, or added to the patch prior to application to the human or animal subject. Gels or emulsions may serve the same purpose of facilitating manufacture by providing a viscous formulation that can be easily manipulated with minimal loss. The term “gel” refers to covalently cross-linked, non cross-linked hydrogel matrices. Hydrogels can be formulated with at least one antigenic agent. Additional excipients may be added to the gel systems that allow for the enhancement of antigen delivery, skin hydration, and protein stability. The term “emulsion” refers to formulations such as water-in-oil creams, oil-in-water creams, ointments, and lotions. Emulsion systems can be either micelle-based, lipid vesicle-based, or both micelle- and lipid vesicle-based.

A liquid formulation may be applied directly to the skin and allowed to air dry or held in place with a dressing, patch, or absorbent material. The formulation may be applied in an absorbent dressing or gauze. The formulation may be covered with an occlusive dressing such as, for example, AQUAPHOR (an emulsion of petrolatum, mineral oil, mineral wax, wool wax, panthenol, bisabol, and glycerin from Beiersdorf), plastic film, COMFEEL (Coloplast) or VASELINE petroleum jelly; or a non-occlusive dressing such as, for example, TEGADERM (3M), DUODERM (3M) or OPSITE (Smith & Napheu).

The relative amount of an antigenic agent within a composition and the dosing schedule can be adjusted appropriately for efficacious administration to a subject (e.g., human or animal). This adjustment may depend on the subject's particular disease or condition, whether therapy or prophylaxis is intended, the administration route, the physical condition and of the subject. To simplify administration of a composition to a subject, each unit dose can contain one or more antigenic agents in predetermined amounts for a single round of immunization. The amount of an antigenic agent in the unit dose can range from about 0.1 μg to about 10 mg.

The compositions of the present invention can be manufactured under good manufacturing practices regulated by government agencies (e.g., Food and Drug Administration) for biologicals and vaccines.

Devices for Transdermal Immunization.

The system of the present invention comprises an apparatus for enhancing transdermal immunization. According to the principles of the present invention the apparatus is used to generate at least one micro-channel in an area on the skin of a subject through which a composition comprising an antigenic agent is delivered efficiently.

The term “micro-channel” as used in the context of the present invention refers to a pathway, generally extending from the surface of the skin through all or significant part of the stratum corneum, through which molecules can diffuse.

According to some embodiments of the present invention, the apparatus for facilitating transdermal movement of an antigenic agent is as disclosed in one or more of the U.S. Pat. Nos. 6,148,232; 6,597,946; 6,611,706; 6,711,435; 6,708,060; and 6,615,079, the contents of which is incorporated by reference as if fully set forth herein. Typically, the apparatus comprises an electrode cartridge comprising a plurality of electrodes, and a main unit comprising a control unit adapted to apply electrical energy to the plurality of electrodes when the electrodes are in vicinity of the skin, typically generating current flow or one or more sparks, enabling ablation of stratum corneum in an area beneath the electrodes, thereby generating at least one micro-channel. The main unit loaded with the electrode cartridge is also denoted herein ViaDerm.

According to some embodiments, the control unit of the apparatus comprises circuitry to control the magnitude, frequency, and/or duration of the electrical energy delivered to the electrodes, so as to control the current flow or spark generation, and thus the width, depth and shape of the one or more formed micro-channels. Preferably, the electrical energy applied by the control unit is at radio frequency (RF).

The micro-channels formed by the apparatus of the present invention are hydrophilic and typically have a diameter of about 10 to about 100 microns and a depth of about 20 to about 300 microns, thus facilitating the diffusion of antigenic agents through the skin.

According to the principles of the present invention, the electrode cartridge comprises a plurality of electrodes thus forming an electrode array, which generates upon application of an electrical energy a plurality of micro-channels within the subject's skin. Typically, however, the overall area of micro-channels generated in the stratum corneum is small compared to the total area covered by the electrode array. For example, the plurality of electrodes may be arranged in an array of from about 80 μm in diameter and 100 μm in lenth. It will be understood that the term “plurality” refers herein to two or more elements, e.g., two or more electrodes or two or more micro-channels.

According to additional embodiments, the pressure obtained while placing the apparatus of the present invention on a subject's skin activates the electrical energy delivered to the electrodes. Such mode of action ensures that activation of electrodes occurs only in a close contact with the skin enabling the desired formation of the micro-channels.

The number and dimension of micro-channels may be adjusted to the amount of the antigenic agent desired to be delivered into the skin. For example, the micro-channels may be present at a density of about 100 micro-channels/cm to about 450 micro-channels/cm of skin. In a further example, the micro-channels may be present at a density of about 150 micro-channels/cm to about 300 micro-channels/cm of skin.

The electrode cartridge is preferably removable. According to certain embodiments, the electrode cartridge is discarded after one use, and as such is designed for easy attachment to the main unit and subsequent detachment from the main unit.

According to the present invention, application of current to the skin causes ablation of at least the stratum corneum, which results in the formation of micro-channels. Spark generation, cessation of spark generation, or a specific current level can be used as a form of feedback, which indicates that the desired depth has been reached and current application should be terminated. For these applications, the electrodes are preferably shaped and/or supported in a cartridge that is conducive to facilitate formation of micro-channels in the stratum corneum to the desired depth, but not beyond that depth. Alternatively, the current can be configured so as to form micro-channels in the stratum corneum without the generation of sparks. The resulted micro-channels are uniform in shape and size.

According to the present invention, the electrodes can be maintained either in contact with the skin, or in vicinity of the skin, up to a distance of about 500 microns therefrom. According to some embodiments, ablation of the stratum corneum is performed by applying electrical current having various frequencies, for example, between about 10 kHz and about 4000 kHz, between about 10 kHz and about 500 kHz, and at about 100 kHz.

The burst length of the electrical current may be about 700 sec to about 13,000 seconds. In further embodiments, the burst length of the electrical current may be about 9,000 seconds.

The starting amplitude of the electrical current may be about 140 V to about 350 V. In further embodiments, the starting amplitude may be at least about 200 V. In a further embodiment, the starting amplitude may be about 290 V.

Methods for Transdermal Immunization.

One embodiment of the present invention further provides a method for inducing an antigen-specific immune response using a transdermal delivery system of the invention. Typically, the procedure for inducing an antigen-specific immune response comprises a step of placing over the skin the apparatus for generating at least one micro-channel. Prior to generating the micro-channels, the treatment sites may be swabbed with pads comprising sterile alcohol. Preferably, the site should be allowed to dry before treatment.

In further embodiments of the present invention, the apparatus containing the electrode array is placed over the site of treatment, the array is energized by RF energy, and treatment is initiated. In principle, the ablation and generation of micro-channels is completed within seconds. The apparatus is removed after micro-channels are generated at limited depth. A composition according to the invention is applied to the area of the treated skin where micro-channels are present.

Another embodiment of the present invention thus provides a method for inducing an antigen-specific immune response by transdermal delivery system comprising the steps of: generating at least one micro-channel in an area of the skin of a subject, and applying a composition comprising an immunogenically effective amount of an antigenic agent to the area of skin in which the at least one micro-channel is present, thereby inducing an antigen-specific immune response.

In another embodiment, a method for transdermally delivering a vaccine to a subject is provided. The method comprises applying a plurality of electrodes to the skin of the subject, generating a plurality of micro-channels through at least the stratum corneum of the subject by generating an electrical current through the skin and delivering a vaccine through the micro-channels.

In a further embodiment, a method for creating micro-channels through the skin of a patient is provided. The method comprises applying a plurality of electrodes to the skin of the subject, generating a plurality of micro-channels in the skin of the subject by generating an electrical current through the skin. The depth of the micro-channels generated may be controlled by the length of the electrodes, the potential difference applied between the plurality of electrodes, the length of time the electrical current is applied to the skin and a combination thereof. In a further embodiment, a method for creating micro-channels through the at least the stratum corneum of a patient is provided. The method comprises applying a plurality of electrodes to the skin of the subject, generating a plurality of micro-channels in the skin of the subject by generating an electrical current through the skin, wherein the electrodes are at least about 30 microns, further at least about 50 microns, further at least about 100 microns.

The term “transdermal” delivery refers to delivery of an antigenic agent into or through the dermal layers of the skin, i.e., the epidermis or dermis, beneath the stratum corneum, or into or through the subcutaneous layers of the skin. Thus, an antigen can be delivered into the skin or through the skin into the blood or lymphatic system. The term transdermal is therefore meant to include also transcutaneous delivery.

The term “immunogenically effective amount” is meant to describe the amount of an antigenic agent, which induces an antigen-specific immune response.

The immune response induced by the composition of the present invention can comprise humoral (i.e., antigen-specific antibody such as IgM, IgD, IgA1, IgA2, IgE, IgG1, IgG2, IgG3, and/or IgG4) and/or cellular (i.e., antigen-specific lymphocytes such as CD4⁺ T cells, CD8⁺ T cells, cytotoxic lymphocytes, Th1 cells, and/or Th2 cells) effector arms. Moreover, the immune response may comprise NK cells that mediate antibody-dependent cell-mediated cytotoxicity (ADCC). The antibody isotypes (e.g., IgM, IgD, IgA1, IgA2, IgE, IgG1, IgG2, IgG3, and IgG4) can be detected by immunoassay techniques as known in the art (see also the Examples herein below) and/or by a neutralizing assay. The terms “inducing an immune response”, “vaccination”, and “immunization” are meant to describe the induction of an immune response, whether humoral or cellular, and are used interchangeably throughout the specification and claims of the present invention.

In a neutralization assay, for example in a viral neutralization assay, serial dilutions of sera are added to host cells, which are then observed for infection after challenge with infectious virus. Alternatively, serial dilutions of sera can be incubated with infectious titers of virus prior to inoculation of an animal, and the inoculated animals are then observed for signs of infection.

The transdermal immunization system of the invention can be evaluated using challenge models in either animals or humans, which evaluate the ability of immunization with an antigenic agent to protect the subject from a disease. Such protection would demonstrate an antigen-specific immune response.

According to the principles of the present invention, induction of an immune response is useful for treating a condition or disease in a subject. Thus, induction of an immune response by the systems and methods of the present invention provides immunoprotection, immunosuppression, modulation of an autoimmune disease, potentiation of cancer immunosurveillance, prophylactic vaccination to prevent disease, and/or therapeutic vaccination to treat or reduce the severity and/or duration of established disease. When the antigen is derived from a pathogen, for example, the treatment may vaccinate the subject against infection by the pathogen or against its pathogenic effects such as those caused by toxin secretion.

A method “induces” an immune response when it causes a statistically significant change in the magnitude or kinetics of the immune response, change in the induced elements of the immune system (e.g., humoral and/or cellular), effect on the number and/or the severity of disease symptoms, effect on the health and well-being of the subject (i.e., morbidity and mortality), or combinations thereof.

It will be appreciated that the application site can be protected with anti-inflammatory corticosteroids or non-steroidal anti-inflammatory drugs (NSAIDs) to reduce possible local skin reaction or modulate the type of immune response. Similarly, anti-inflammatory steroids or NSAIDs can be included in the patch material, in creams, ointments, and a like or alternatively corticosteroids or NSAIDs may be applied after application of the formulation of the invention. IL-10, TNF-α, or any other immunomodulator can be used instead of the anti-inflammatory agents. Alternatively or additionally, pimecrolimus, tacrolimus, aloevera or any other agent known in the art to reduce local skin reaction can be applied to the treated skin area or included in the patch.

Vaccination has also been used as a treatment for cancer and autoimmune diseases. For example, vaccination with a tumor antigen (e.g., prostate specific antigen) can induce an immune response in the form of antibodies, CTLs and lymphocyte proliferation, which allows the body's immune system to recognize and kill tumor cells. Tumor antigens useful for vaccination have been described for melanoma, prostate carcinoma, and lymphoma.

Vaccination with T-cell receptor oligopeptide can induce an immune response that halts the progression of autoimmune disease. U.S. Pat. No. 5,552,300 describes antigens suitable for treating autoimmune disease.

It is to be understood that transdermal immunization may be followed with enteral, mucosal, and/or other parenteral techniques for boosting immunization with the same or altered antigens. Immunization by an enteral, mucosal, and/or other parenteral route may be followed with transdermal immunization for boosting immunization with the same or altered antigens.

### EXAMPLES

Transdermal vaccination using an apparatus that generates micro-channels in the skin of a subject, which is denoted herein ViaDerm, was compared to the widely used subcutaneous (SC) and intramuscular (IM) vaccination routes in order to establish its usefulness as a potential vaccine administration system.

Ovalbumin (OA) and trivalent influenza virus (TIV) were used as exemplary antigens to establish the efficacy of the system of the present invention to induce antigen-specific immune response.

### Example 1

### Transdermal Immunization with Ovalbumin

Materials. A solution of ovalbumin (50 μg/ml water; Sigma) was used for IM and SC injections.

A solution of ovalbumin (10 mg/ml) was used for solution transdermal administration (VD-s).

Ovalbumin powder (2 mg) was used for powder transdermal administration (VD-p).

A solution pouch was prepared as follows: a 300 μm thick layer of adhesive (Durotac 2516, National starch, Netherlands) was evenly spread over a silicone sheet (Sil-k Degania Silicone, Israel). The sheet was cut into 4×4 cm squares. A square hole (1.57×1.57 cm) was cut in the middle of each of the 4×4 squares. A piece of Sil-k silicone 2×2 cm is adhered to the 4×4 cm silicone square over the 1.57×1.57 cm hole using 7701 primer and 4011 glue (Loctite, Ireland). The final product was a pouch of 250 μl volume.

Powder patch was prepared as follows: ovalbumin powder was distributed on the skin and then covered with a fixing patch containing BLF 2080 liner (Dow, USA) covered with a layer of Durotak 2516 adhesive (National starch, Netherlands) or alternatively with Tegaderm™ (3M).

Procedure. Blood was collected intracardially or by abdominal Vena Cava venipuncture immediately prior to immunization and at weekly intervals starting 8 days post immunization. Each sample contained 1.3 ml of blood in Heparin anticoagulant tubes. The blood samples were centrifuged at 6000 rpm and the plasma was collected.

Group 1: Intramuscular injection. Guinea pigs, males, 600-650 gr, Dunkin Hartley (7 animals) were anesthetized and blood (1.3 ml) was collected immediately prior to immunization. Ovalbumin solution was then injected (5 μg; 0.1 ml of 50 μg/ml) to the Quadriceps muscle of the right hind leg. Blood was drawn from each animal at days 8, 15, 22, and 30 after immunization. At day 30, the animals were injected again to the Quadriceps muscle of the right hind leg (boost-5 μg; 0.1 ml of 50 μg/ml). Blood was collected at days 36, 42, 50, and 125 days after immunization.

Group 2: Subcutaneous immunization. Guinea pigs, males, 600-650 gr, Dunkin Hartley (7 animals) were anesthetized and blood (1.3 ml) was collected immediately prior to immunization. Ovalbumin solution was then injected (5 μg; 0.1 ml of 50 μg/ml) subcutaneously to the dorsal neck area. Blood was drawn from each animal at days 8, 15, 22, and 30 after immunization. At day 30, the animals were injected again (boost-5 μg; 0.1 ml of 50 μg/ml) subcutaneously to the dorsal neck area. Blood was collected at days 36, 42, 50, and 125 days after immunization.

Group 3: Transdermal immunization by application of an ovalbumin solution pouch to ViaDerm treated skin. Guinea pigs, males, 600-650 gr, Dunkin Hartley (7 animals) were anesthesized and blood (1.3 ml) was collected immediately prior to immunization. The animals were treated with a device, denoted herein ViaDerm, which utilizes electrical energy at radio frequency and consists of an array of electrodes, to generate micro-channels in the skin of the guinea pigs (see, for example, WO 2004/039426; WO 2004/039427; and WO 2004/039428 incorporated by reference as if fully set forth herein). ViaDerm Operating Parameters: burst length (μsec)—700; starting amplitude—330V; number of bursts—5; 2 applications on the same skin area (200 pores/cm²). Ovalbumin solution pouch (2 mg; 0.2 ml of 10 mg/ml) was placed on the treated skin area. Twenty-four hours post application, the pouch was removed. Blood was drawn from each animal at days 8, 15, 22, and 30 after immunization. At day 30, the animals were immunized again by ViaDerm treatment as described above, i.e., burst length (μsec)—700; starting amplitude—330V; number of bursts—5; 2 applications on the same skin area (200 pores/cm²), followed by transdermal application of an ovalbumin solution pouch (2 mg; 0.2 ml of 10 mg/ml). Blood was collected at days 36, 42, 50, and 125 days after immunization.

Group 4: Transdermal immunization by application of an ovalbumin powder patch to ViaDerm pretreated skin. Guinea pigs, males, 600-650 gr, Dunkin Hartley (7 animals) were anesthesized and blood (1.3 ml) was collected immediately prior to the immunization. The animals were treated with ViaDerm. ViaDerm Operating Parameters: burst length (μsec)—700; starting amplitude—330V; number of bursts—5; 2 applications on the same skin area (200 pores/cm²). Ovalbumin powder (2 mg) was evenly distributed with a spatula on the treated skin area and then covered with a fixing patch. Twenty-four hours post application, the patch was removed. Blood was drawn from each animal at days 8, 15, 22, and 30 after immunization. At day 30, the animals were immunized again by ViaDerm treatment as described above, i.e., burst length (μsec)—700; starting amplitude—330V; number of bursts—5; 2 applications on the same skin area (200 pores/cm²), followed by transdermal application of ovalbumin powder (2 mg; 0.2 ml of 10 mg/ml) as described above. Blood was collected at days 36, 42, 50, and 125 days after immunization.

Detection of anti-ovalbumin antibodies in guinea-pig plasma samples: Ninety six-well plates (Maxisorp; Nunc, Denmark) were coated with ovalbumin (100 μl of a solution of 200 μg/ml). Coating was conducted for 16-18 hours at 4° C. Unbound ovalbumin was removed by washing three times with a wash solution (PBS containing 0.05% Tween 20). Remaining adsorption sites were blocked with a diluent/blocker solution (PBS containing 0.05% Tween 20 and 4% skim milk) for one hour at room temperature, followed by three washes with the wash solution.

Guinea pig's plasma samples, serially diluted with the diluent/blocker, were added to the ovalbumin-coated plates in triplicates and incubated for one hour at 22° C. Unbound antibodies were washed three times with the wash solution. In order to detect guinea pig IgG antibodies, the wells were incubated for one hour at 22° C. with horseradish-peroxidase (HRP) conjugated goat-anti guinea pig IgG antibody diluted in the diluent/blocker solution (Jackson Immunoresearch Laboratories, 0.8 mg/ml, 1:10,000), and then washed three times with the wash solution. In order to detect IgA or IgM guinea pig antibodies, the wells were incubated for one hour at 22° C. with rabbit anti-guinea pig IgA or rabbit anti-guinea pig IgM, respectively (both were purchased from I.C.L; 1:5,000 dilution). Unbound antibodies were washed three times with the wash solution. Then, horseradish-peroxidase (HRP) conjugated donkey anti-rabbit IgG diluted in the diluent/blocker solution (Jackson Immunoresearch laboratories; 1:5,000) was incubated for one hour at 22° C., followed by three washes as described above.

HRP substrate (Substrate-chromogen, TMB-ready to use, DAKO) was then added and incubated for 30 minutes at 22° C. The reaction was stopped with 1 M H₂SO₄.

The signal was detected in a spectrophotometer at 405 nm and the background at 595 nm.

Titer Calculation: The average (AVG) optical density (O.D.) data was calculated for every duplicate/triplicate of the samples. Similarly, AVG O.D.s were obtained from equivalent dilutions of normal plasma samples (from naive non-immunized animals). The AVG O.D.s obtained from non-immunized animals were subtracted from the O.D.s obtained from the immunized animals.

The data obtained for an internal standard (animal #27 at day 36) was plotted in a logarithmic scale. Using this plot, the linear-power regression range was determined. The end point titer (titer) is calculated using the regression formula obtained from the linear range. The cut-off O.D. (y axis-“noise” cut-off) data was calculated as 5 times blank STD.

Results. Trans epidermal water loss (TEWL; DERMALAB® Cortex Technology, Hadsund, Denmark) measurements were used to verify the efficacy of micro channel formation by measuring TEWL levels on potential treatment sites before ViaDerm application (BVD) and after ViaDerm application (AVD). Only sites that were within the TEWL specification (i.e., TEWL before treatment ≦8.5 g/h/m²; Δ TEWL ≧20 g/h/m²) were approved for testing. The results are presented in Tables 1 and 2.

TABLE 1
TEWL of primary immunization.

| | | TEWL | |
|---|---|---|---|
| | Guinea | BVD | TEWL AVD |
| Group | Pig | (g/h/m²) | (g/h/m²) |
| Transdermal, 2 mg | 15 | 3.1 | 46 |
| OVA solution | 16 | 5.3 | 34.9 |
| | 17 | 4.3 | 39 |
| | 18 | 5 | 36.5 |
| | 19 | 4.9 | 37.5 |
| | 20 | 4.9 | 40.8 |
| | 21 | 4.6 | 47.6 |
| | AVG | 4.59 | 40.33 |
| | STDEV | 0.73 | 4.82 |
| Transdermal, 2 mg | 22 | 5.7 | 44.9 |
| OVA powder | 23 | 5.1 | 33.7 |
| | 24 | 6.1 | 36.9 |
| | 25 | 5.5 | 35.8 |
| | 26 | 4.7 | 41.7 |
| | 27 | 6.3 | 38.3 |
| | 28 | 4.3 | 46.9 |
| | AVG | 5.39 | 39.74 |
| | STDEV | 0.73 | 4.90 |

TABLE 2
TEWL of boost immunization.

| | | TEWL | |
|---|---|---|---|
| | Guinea | BVD | TEWL AVD |
| Group | Pig | (g/h/m²) | (g/h/m²) |
| Transdermal, 2 mg | 15 | 5.7 | 44.9 |
| OVA solution | 16 | 5.5 | 38.7 |
| | 17 | 5.8 | 34.9 |
| | 20 | 5 | 43.8 |
| | 21 | 3.8 | 39.8 |
| | AVG | 5.16 | 40.42 |
| | STDEV | 0.82 | 4.04 |
| TD, 2 mg OVA | 22 | 4.2 | 34.2 |
| powder | 23 | 2.7 | 33.1 |
| | 25 | 3 | 24.7 |
| | 26 | 1.6 | 33.5 |
| | 27 | 0.8 | 31.1 |
| | 28 | 3.8 | 38.7 |
| | AVG | 2.68 | 32.55 |
| | STDEV | 1.29 | 4.59 |

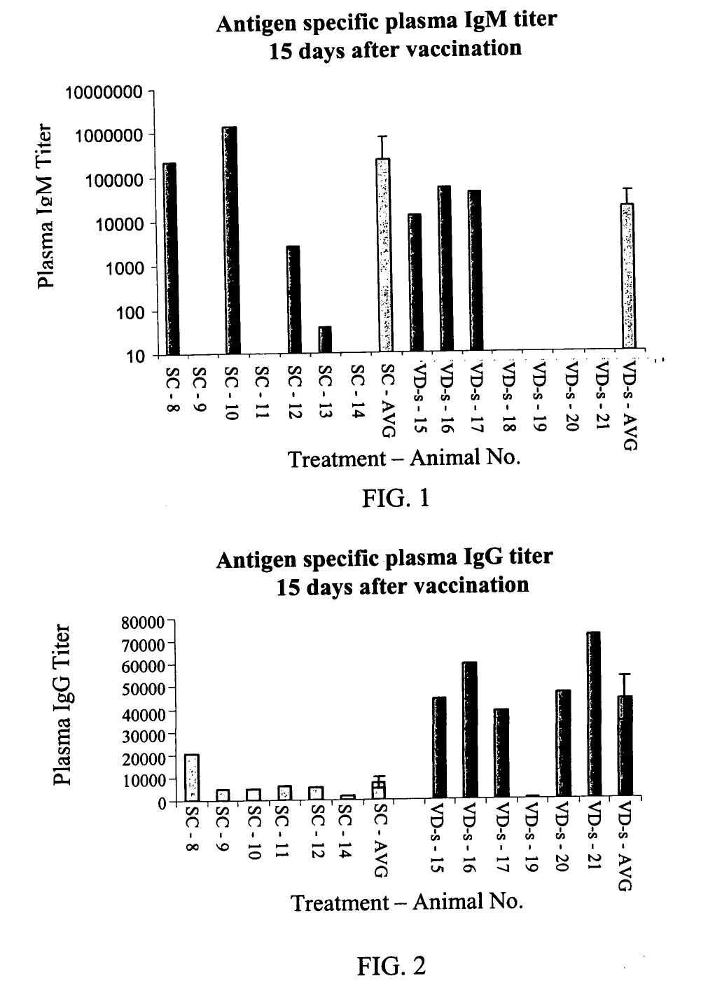

IgM. IgM antibodies 15 days after primary immunization represent the earliest response to antigen presentation. As shown in FIG. 1, the group of animals injected subcutaneously (SC) with ovalbumin and the group of animals treated with ViaDerm and thereafter immunized against ovalbumin by the ovalbumin solution pouch (VD-s) showed induction of ovalbumin specific IgM antibodies, though the IgM antibody titer detected in the SC group was higher than in the VD-s group. In addition, both groups demonstrated similar incidence of “non-responder” animals, e.g., animals that did not show detectable titer of IgM antibodies.

IgG: The appearance of antigen specific IgG antibodies following antigen presentation express the maturation of the antigen specific immune response.

FIG. 2 presents the IgG plasma titers 15 days post immunization. There was a significant difference between the VD-s group and the SC group. As shown in FIG. 2, generation of micro-channels by ViaDerm treatment and subsequent application of the ovalbumin solution pouch (VD-s) resulted in significantly higher IgG titers at day 15 compared to the titers obtained by SC injection. These results clearly indicate that ViaDerm treatment can shorten the time for IgG antibodies appearance. This effect is highly advantageous as IgG antibodies are the most important antibody subtype in an antigen specific immune response.

FIG. 2 also shows that all the animals in the VD-s and SC groups were found to be positive for antigen specific plasma IgG antibodies. FIG. 2 further shows that there was low variability between the individual animals. The single animal of the VD-s group that did not show detectable titer of IgG (Animal No. 19) was found to be in a bad physical condition at the time of bleeding and died the next day. Animal No. 19 did not have any detectable antigen specific IgM and IgA.

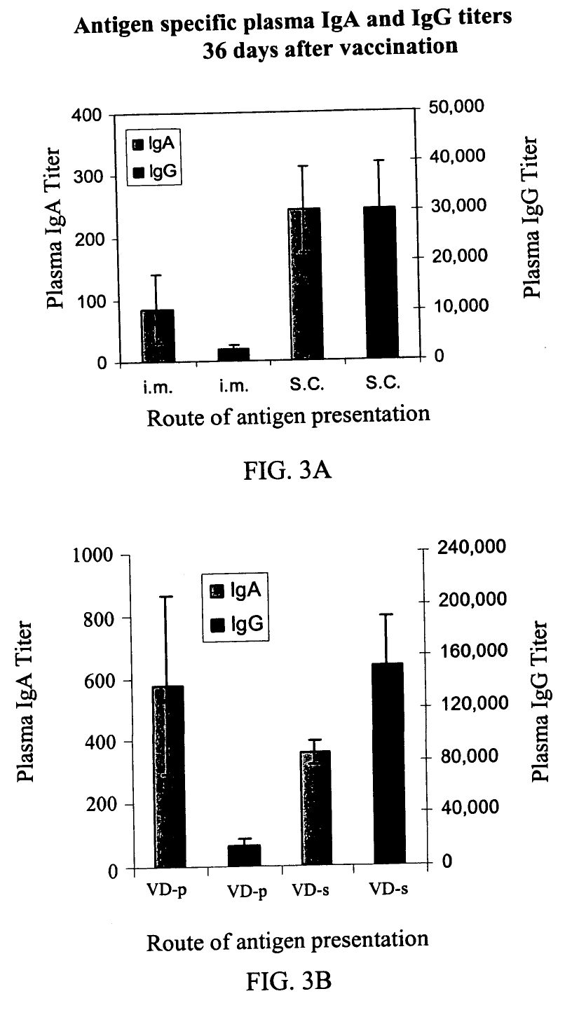

Six days after the boost (FIG. 3), there was a strong IgG antibody secondary response in both the VD-s and the SC groups, with plasma titers that were 3.5 and 4.1 greater (for VD-s and SC, respectively) over the titers observed 15 days post immunization. It should be also noted that the IgG titers in the VD-s group were approximately 5 times higher than in the SC group, indicating the efficacy of this method in eliciting an antigen specific IgG antibodies. The IgG titers in the intramuscularly (IM) injected group were very low compared to all other groups, including the SC group immunized with the same ovalbumin dose.

A comparison of the two transdermal formulations revealed that the IgG titer for the VD-s was 9.5 times greater than the VD-p group (using the same dose). The IgG titer for the VD-p group was lower than that of the SC group, which received a lower dose.

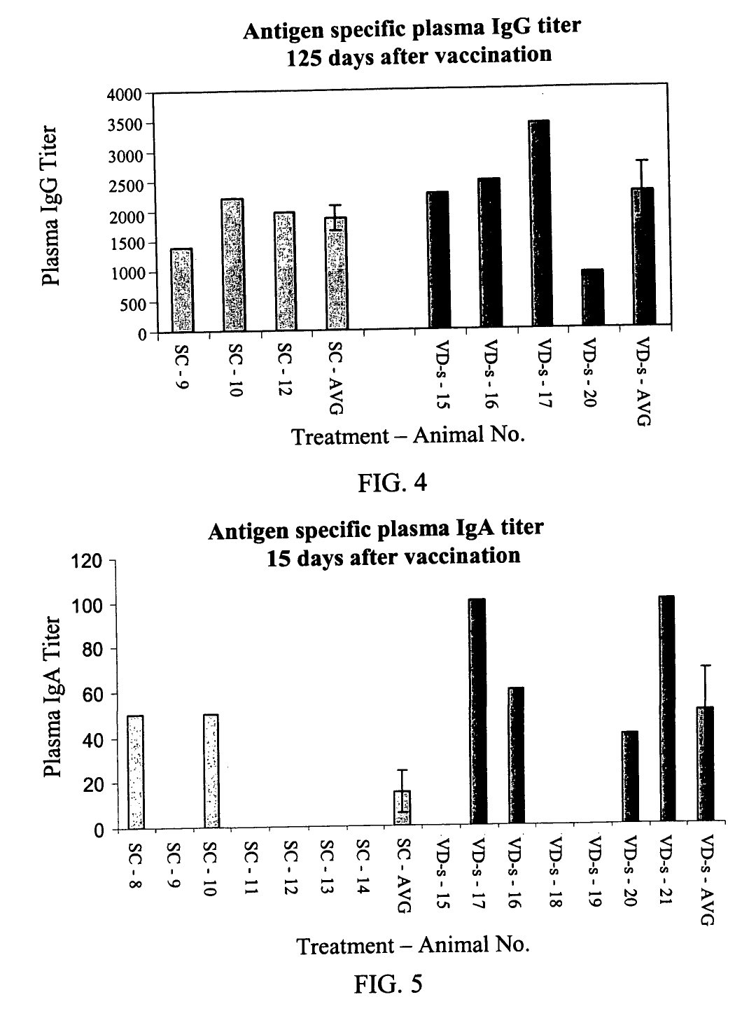

Ninety-five days after boost administration (FIG. 4), only 1.3% and 6% of the IgG antibody titer were detected in the VD-s and SC groups, respectively.

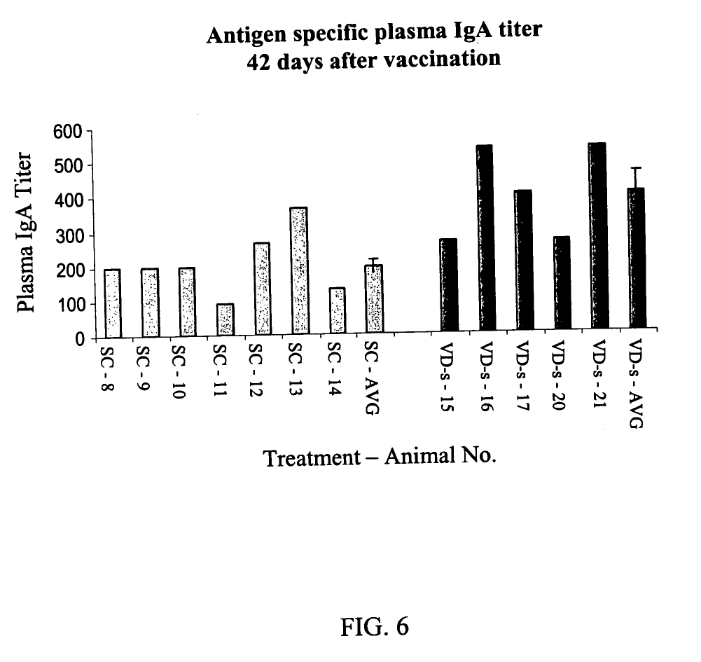

IgA: The antigen specific plasma IgA titer was determined in the SC and the VD-s groups at 15 days post primary antigen presentation (FIG. 5). Only 2 out of 7 animals in the SC group demonstrated detectable IgA titers compared to 4 animals out of 6 in the VD-s group. This superiority of the VD-s treatment compared to the SC injection was further demonstrated six days after boost administration (FIG. 6). The animals that had no detectable specific IgA response after boost administration (animals Nos. 9&11) had neither IgA nor IgM at 15 days post immunization. All animals (SC and VD-s) were IgA positive 12 days after antigen boosting (FIG. 6).

The higher titers in VD groups as well as the high frequency of sero-positive individual animals indicate the usefulness of transdermal immunization using ViaDerm. The time for appearance of significant titers of IgG and IgA antibodies was shorter in the ViaDerm treated groups compared to that of the well-established and widely accepted SC and IM routes, thus indicating the efficacy of this transdermal route of immunization.

The significant immune response following VD antigen presentation included all the important plasma antibody isotypes: IgM, IgG and IgA, thus indicating efficient isotype switching. There was no correlation between IgG and IgM antibody titers in the VD-s vs. S.C. groups. Thus, while higher IgG titers were observed in the VD-s group vs. SC group, higher IgM titers were observed in the SC group vs. VD-s group during the primary response. Without being bound to any theory, this phenomenon may be explained by a very efficient cellular response, which takes place following VD application. This data is supported by previous observations performed by the applicant of the present invention demonstrating that shortly after VD application there is a strong leukocyte infiltration around the micro-channels, providing an adjuvant-like effect. As isotype switching is a process involving antigen presentation and extended support by T helper lymphocytes existing mainly in the peripheral lymph nodes (PLN), it can be speculated that VD treatment can activate local “professional” antigen presenting dendritic cells (APDC). Shortly after VD antigen presentation, APDC can activate lymphocytes locally, following their infiltration to the inflamed micro channel site. Yet, it will be understood that the majority of these interactions are normally taking place in the PLN, the natural target for activated APDC migration.

While the route of antigen presentation is an important parameter for inducing antigen-specific immune response, the use of antigen-formulation and adjutants can be equally important. In the present example, VD treatment was used with two ovalbumin formulations, i.e., powder (VD-p) and solution (VD-s), at the same dose and in the absence of any adjuvant. The lower IgG titer in the VD-p vs. VD-s emphasized that antigen-formulation is critical for successful vaccine development. The impressive IgA titer in the VD-p group compared to the poor IgG titer strongly indicates that antigen formulation can play a significant role in manipulating the immune response as desired. Because specific antibody isotypes are often more important than others in a given condition, it can be very useful to utilize this phenomenon. For example, in diseases of mucous membranes application of a dry antigen with the apparatus of the present invention can be advantageous in order to elicit IgA antibodies, which are secreted from these membranes.

Thus, transdermal immunization using ViaDerm technology is highly efficient and can provide an alternative technique for the traditional vaccination routes.

### Example 2

### Transdermal Immunization with Trivalent Influenza Vaccine

Materials. Female Hartley guinea pigs (>350 g), >7 weeks old (Charles River).

Inactivated influenza vaccine: A/Panama/2007/99, A/New Caledonia/20/99 and B/Shangdong/7/97, lot#001, 2.046 mg/ml, diluted to 0.2046 mg/ml for use.

E. coli heat labile enterotoxin (LT): FIN0023, 1.906 mg/ml.

One-layer rayon square patch 1 cm².

ViaDerm: Length of electrodes 30 and 100 μm, cylinder shape and 50 μm, conic shape.

Tegaderm 1624W: 3M, NDC 8333-1624-05, 6 cm×7 cm size

Adhesive tape: 3M

Hydration solution: 10% Glycerol/saline

Immunization. Before immunization, the guinea pigs were shaved and sedated with ketamine and xylazine. All animals were bolus intramuscular injected with 0.5 μg HA (0.17 μg HA each strain) in 100 ul 1×DPBS on study day 1.

Pretreatment. Guinea pigs were shaved on the abdomen one day before immunization and re-shaved immediately before patch application on study day 22. The immunization site was marked with a permanent marker and the shaven skin was pretreated as follows:

Groups 1-2 were hydrated with 10% glycerol/saline;

Groups 3-4 were pretreated with the ViaDerm device <50 μm twice on dry, shaven skin hydrated with 10% glycerol/saline after ViaDerm pretreatment;

Groups 5-6 were pretreated with the ViaDerm device <50 μm twice on dry, shaven skin without hydration; and

Groups 7-8 were pretreated with the ViaDerm device 100 μm twice on dry, shaven skin without hydration.

TEWL measurements were taken before and immediately following pretreatment as known in the art (see, for example, WO 2004/039426; WO 2004/039427; and WO 2004/039428).

Patch application A 1 cm² rayon patch containing 15 μg HA (5 μg HA each strain) alone (no LT) or with 1 μg LT in 15 μl 1×DPBS were applied immediately after the pretreatment. To ensure proper patch adherence, patches were covered with a modified Tegaderm overlay. The patch was wrapped with adhesive tape. Patches were applied for 18-24 hr, removed, and the skin was rinsed with warm water.

Serum collection. Pre-immune (prior to immunization) and post immune (day 22 and 36) blood samples were collected from the orbital plexus using standard methods. Serum was collected by centrifugation of whole blood and the cell free serum transferred to a labeled tube and stored frozen at −20° C.

ELISA. Sera was evaluated for total IgG titers to A/Panama, A/New Caledonia, and B/Shangdong using an ELISA method known in the art (see, for example, US Patent Application Publication No. 2004/018055 incorporated by reference as if fully set forth herein). Antibody titers were presented as ELISA Units (EU), which is the serum dilution equal to 1 O.D. at 405 nm.

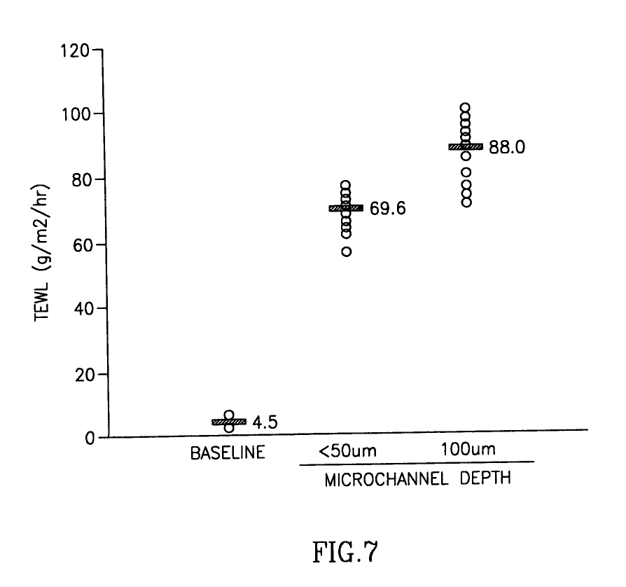

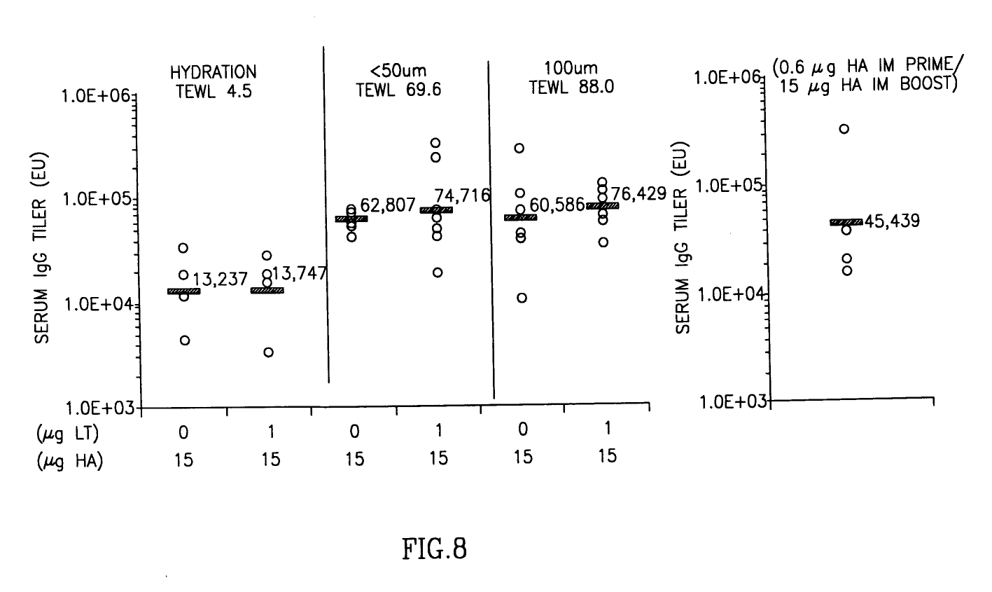

Results. FIG. 7 shows the TEWL values of non-treated or ViaDerm treated guinea pigs. As shown in FIG. 7, TEWL values obtained in guinea pigs treated with 100-micron length electrodes of ViaDerm or 50-micron length electrodes of ViaDerm were significantly higher than those obtained from non-treated guinea pigs. These results confirm that micro channels were generated in the skin of the guinea pigs.

FIG. 8 shows serum IgG antibody titers against A/Panama influenza strain in the absence or presence of E. coli heat labile enterotoxin (LT) as an adjuvant in guinea pigs treated with ViaDerm and immunized by a patch containing the trivalent influenza vaccine. As shown in FIG. 8, ViaDerm treatment of guinea pigs either with 50-micron or 100-micron length electrodes followed by influenza patch application significantly increased the IgG antibody titers against A/Panama influenza strain as compared to guinea pigs, which were not treated with ViaDerm but administered with influenza patch. Addition of LT as an adjuvant did not improve the IgG antibody titer against this strain of influenza. As a comparison, guinea pigs were immunized intramuscularly (IM) with 0.5 μg of the trivalent influenza vaccine at day 1, and boosted IM with the same vaccine (15 μg) at day 22. As shown in FIG. 8, the IgG antibody titers in the ViaDerm treated groups were comparable, or even higher, than those of the IM injected guinea pigs, indicating that transdermal immunization using ViaDerm is as efficient as IM immunization.

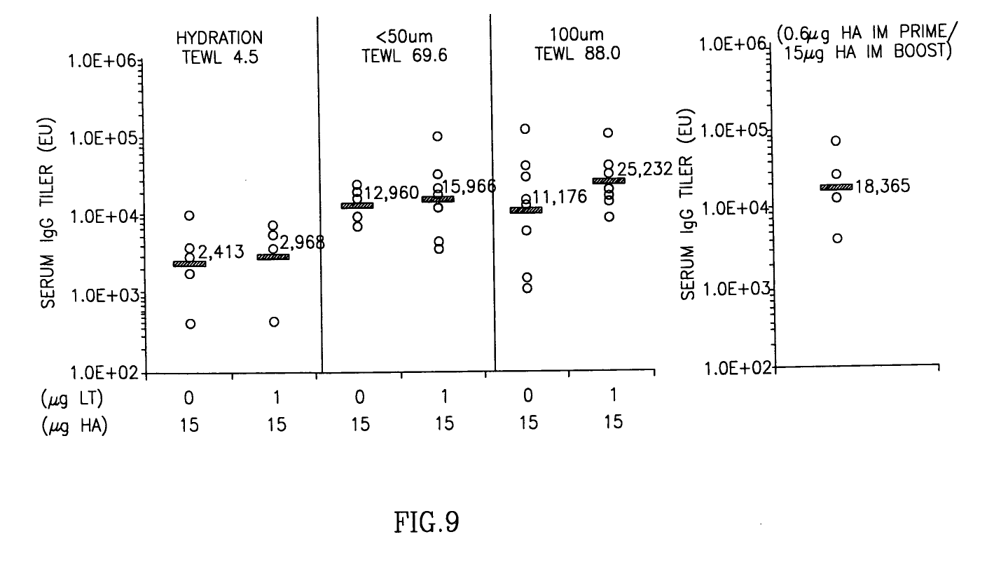

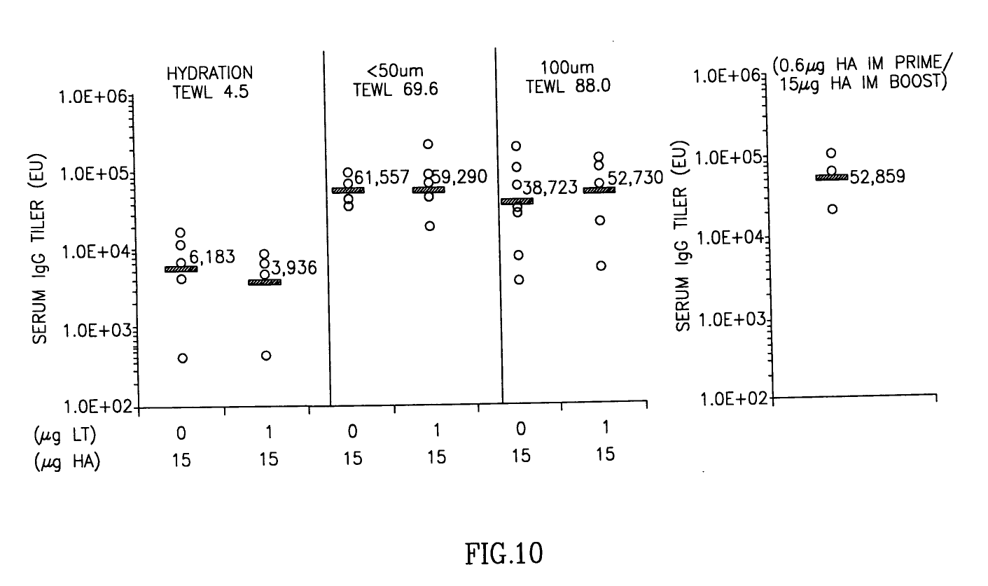

FIGS. 9 and 10 show similar results when serum IgG antibody titers against A/New Caledonia strain and B/Shangdong strain of influenza were determined. As shown in FIGS. 9 and 10, the IgG antibody titers against each of these strains was significantly higher in the ViaDerm treated guinea pigs that were then administered with the influenza patch as compared to guinea pigs not treated with ViaDerm but administered with the influenza patch. Addition of LT as an adjuvant did not improve the IgG antibody titers. The IgG antibody titers in ViaDerm treated animals were comparable to those obtained in guinea pigs injected intramuscularly with the trivalent influenza vaccine.

### Example 3

### Transdermal Immunization with Trivalent Influenza Vaccine

Materials. 47 female Hartley guinea pigs, >350 g, >7 weeks old (Charles River).

Inactivated influenza vaccine: A/Wyoming/03/2003 (H3N2), lot#1028825-0012, 284 ugHA/ml; A/New Caledonia/20/99 (H1N1), lot#1028827-0016, 237 ugHA/ml; B/Jingsu/10/2003, lot#1028826-0009, 542 ugHA/ml.

LT: FIN0023, 1.906 mg/ml.

Dry rayon patch at 1 cm²

ViaDerm: Length of electrodies at <50 um, cylinder shape

Tegaderm 1624W: 3M, NDC 8333-1624-05, 6 cm×7 cm size

Adhesive tape: 3M

Immunization. Before immunization and shaving, guinea pigs were be sedated with ketamine and xylazine by standard procedure. All animals were bolus intramuscular injected with 0.5 μg HA (0.17 μg HA each strain) in 100 μl 1×DPBS on study day 1.

Pretreatment: Guinea pigs were shaved on the abdomen immediately before patch application on study day 22. The immunization site was marked with a permanent marker and the shaven skin was pretreated as follows:

Groups 1-2 were hydrated with 10% glycerol in saline without rubbing;

Groups 3-4 were be pretreated with a ECG prep pad at 15 strokes on 10% glycerol in saline hydrated skin;

Groups 5-6 abdominal area was be cleaned with 70% Ispopronal pad for 10 minutes air-dry, then pretreated with 2-applications with Viaderm within approximately a 1 cm² area on dry skin. No additional hydration with 10% glycerol in saline after TEWL determination.

Groups 7-8 abdominal area were cleaned with 70% Ispopronal pad for 10 minuets air-dry, then pretreated with 2-applications with Viaderm within approximately a 1 cm² area on dry skin. Additional hydration with 10% glycerol/saline after TEWL determination.

Groups 10 were treated with 4-applications with the Viaderm within approximately a 1 cm² area. The treated area was be excised and fixed in formalin.

Patch application. A 1 cm² dry patch was applied to the pretreated skin in the following groups 1-8:

Groups 1, 3, 5 and 7 were dosed with 15 μg HA (0.5 μg per strain) alone

Groups 2, 4, 6 and 8 were dosed with 15 μg HA mixed with 5 μg LT.

Groups 9 were boosted by intramuscular (IM) injection with 15 μg HA.

To insure proper patch adherence, patches were covered with a modified Tegaderm overlay. Animals were wrapped with adhesive tape. Patches were applied for 18-24 hr, removed, and the skin rinsed with warm water.

Serum collection. Pre-immune and post immune (day 36) blood samples were collected from the orbital plexus using standard methods. Serum was collected by centrifugation of whole blood and the cell free serum transferred to a labeled tube and stored frozen at −20° C.

ELISA. Sera was evaluated for total IgG titers to A/Wyoming, A/New Caledonia, B/Jiangsu and LT using an ELISA method. Antibody titers were reported as ELISA Units (EU), which is the serum dilution equal to 1OD at 405 nm. Student T test will be used.

Results. TEWL values obtained in guinea pigs treated with 50 micron length electrodes of ViaDerm (41.1) were significantly higher than those obtained from non-treated guinea pigs (5.3), but below those obtained from guinea pigs treated with ECG pad (63.3). These results confirm that microchannels were generated in the skin of guinea pigs.

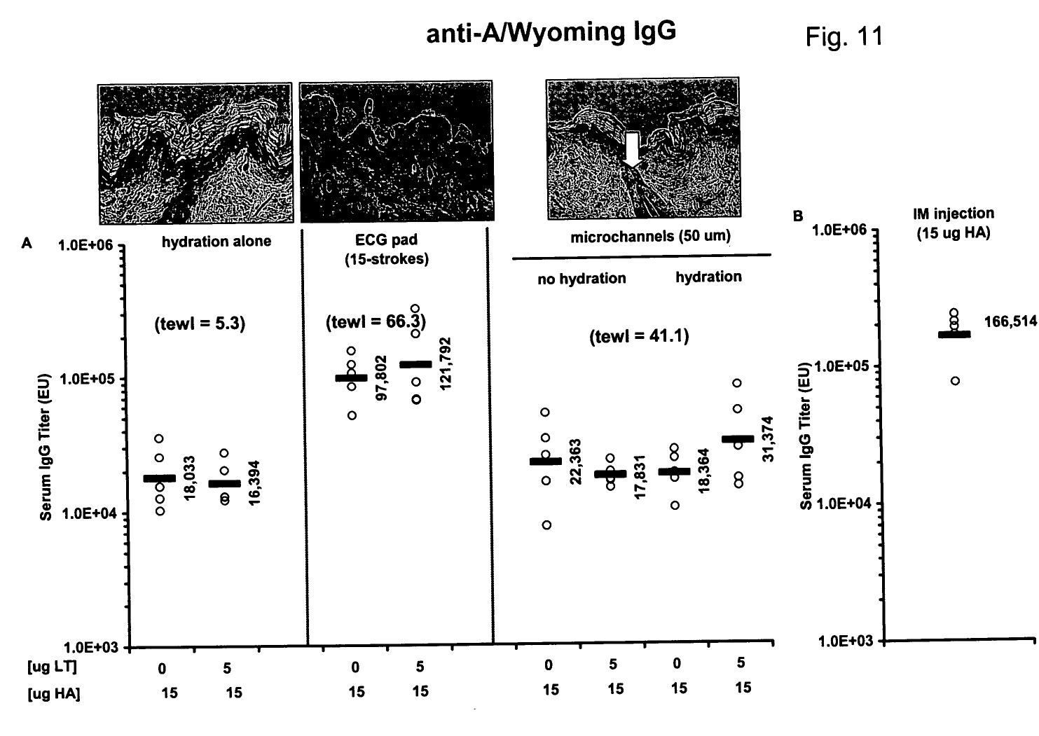

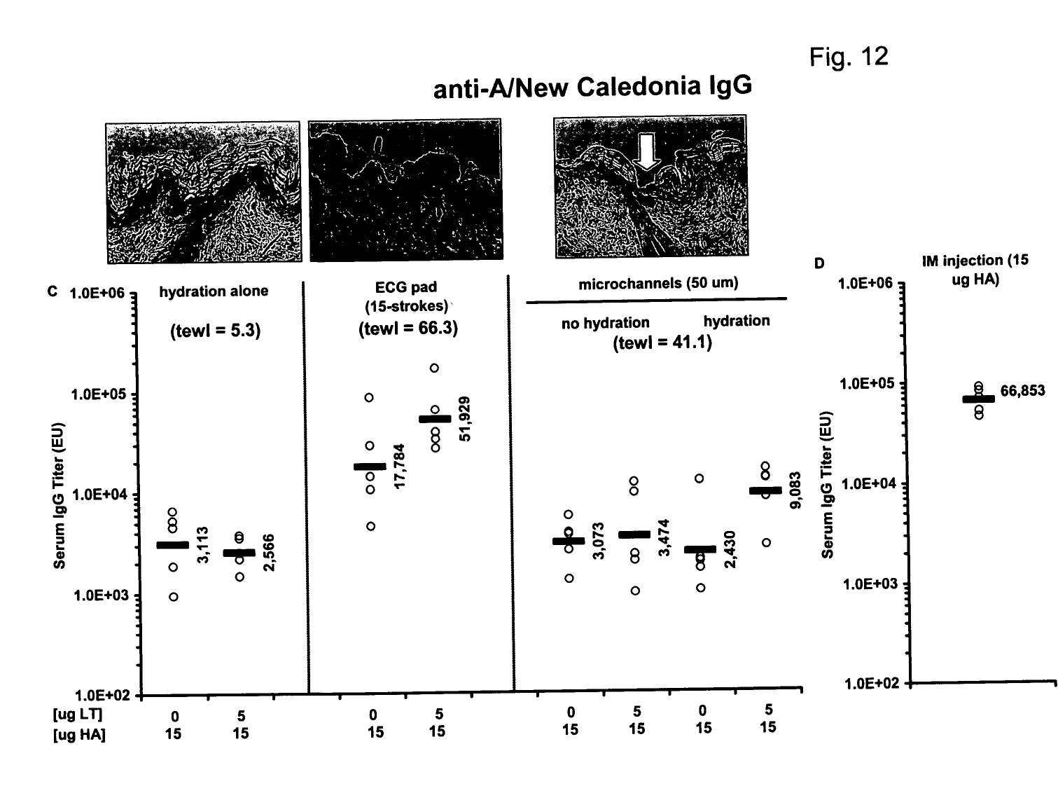

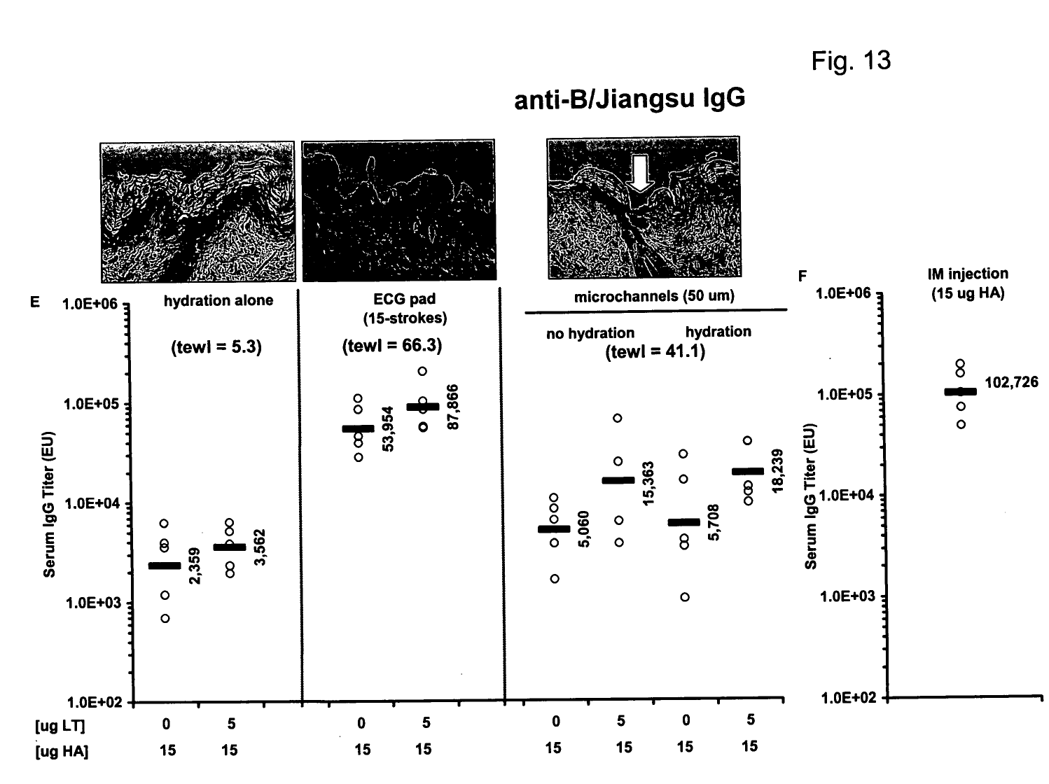

As depicted in FIG. 11, serum IgG antibody titers against A/Wyoming strain in guinea pigs was measured. In guinea pigs treated with the ViaDerm device, the IgG antibody titers against A/Wyoming were significantly higher than those obtained from guinea pigs not treated with the ViaDerm device, but instead were treated with the patch on intact skin. Hydration in addition to treatment with the device also significantly increased the IgG antibody titer as compared to guinea pigs treated with the device having no hydration for A/New Caledonia with adjuvant. Similar results were obtained when serum IgG antibody titers against the A/New Caledonia and A/Jiangsu strains were determined, as shown in FIG. 12 and FIG. 13.

### Example 4

### Immune Response to Flue Vaccine Formulated in a Dry Patch

Materials. 56 female Hartley guinea pigs, >350 g, >7 weeks old (Charles River).

Inactivated influenza vaccine: A/Wyoming/03/2003 (H3N2), lot#1028825-0012, 284 ugHA/ml; A/New Caledonia/20/99 (H1N1), lot#1028827-0016, 237 ugHA/ml; B/Jingsu/10/2003, lot#1028826-0009, 542 ugHA/ml.

LT lot # Fin 0023.

1 cm2 rayon patch, formulated in dried sucrose containing 15 μg HA (5 μg per strain) alone or with LT 5 μg. (Sucrose 12%, Trehalose 8% PVP 0.5%, Pluronic F68 0.1%).

ViaDerm: Electrodes at a depth at <50 um, cylinder shape

IDEO mask/abrade (M&A) device (180 grit sandpaper)

Tegaderm 1624W: 3M, NDC 8333-1624-05, 6 cm×7 cm size

Adhesive tape: 3M

Immunization. Before immunization and shaving, guinea pigs were sedated with ketamine and xylazine by standard procedure. All animals were bolus intramuscular injected with 0.5 μg HA (0.17 μg HA each strain) in 100 μl 1×DPBS on study day 1.

Pretreatment. Hairy guinea pigs were shaved on the abdomen immediately before patch application on study day 22. The immunization site was marked with a permanent marker and the shaven skin was pretreated as follows:

Groups 1-4 were pretreated with an IDEO mask/abrade device (5 times) within a 1 cm² area on dry skin. Tewl measurements were determined after 5 min air-dry of pretreatment. The skin was hydrated with 10% glycerol/saline immediately before patch application.

Groups 5-8 were pretreated with 2-applications with the Viaderm device applied within a 1 cm² area on dry skin. Tewl measurements were determined right following pretreatment. The skin will be hydrated with 10% glycerol/saline immediately before patch application.

Patch application. To insure proper patch adherence, patches were covered with a modified Tegaderm overlay. Animals were wrapped with adhesive tape. Patches were applied for 18-24 hr, removed, and the skin rinsed with warm water.

Groups will be dosed as follows:

Groups 1 and 5 were immunized using a silicone chamber filled with 15 ug HA in ˜50 μl. The chamber was sealed with Tegaderm.

Groups 2 and 6 were immunized using a silicone chamber filled with 15 ug HA+LT in ˜50 μl. The chamber was sealed with Tegaderm.

Groups 3 and 7 were immunized using dry sucrose patches (1 cm²) containing 15 ug HA.

Groups 4 and 8 were immunized using dry sucrose patches (1 cm²) containing 15 ug HA+LT.

Group 9 was boosted by intramuscular (IM) injection with 15 μg HA on day 22.

Serum collection. Post immune (day 21 and 36) blood samples were collected from the orbital plexus using standard methods. Serum was collected by centrifugation of whole blood and the cell free serum transferred to a labeled tube and stored frozen at −20° C.

ELISA: Sera was evaluated for total IgG titers to A/Wyoming, A/New Caledonia, B/Jiangsu and LT using an ELISA method. Antibody titers were reported as ELISA Units (EU), which is the serum dilution equal to 1OD at 405 nm. Student T test was used.

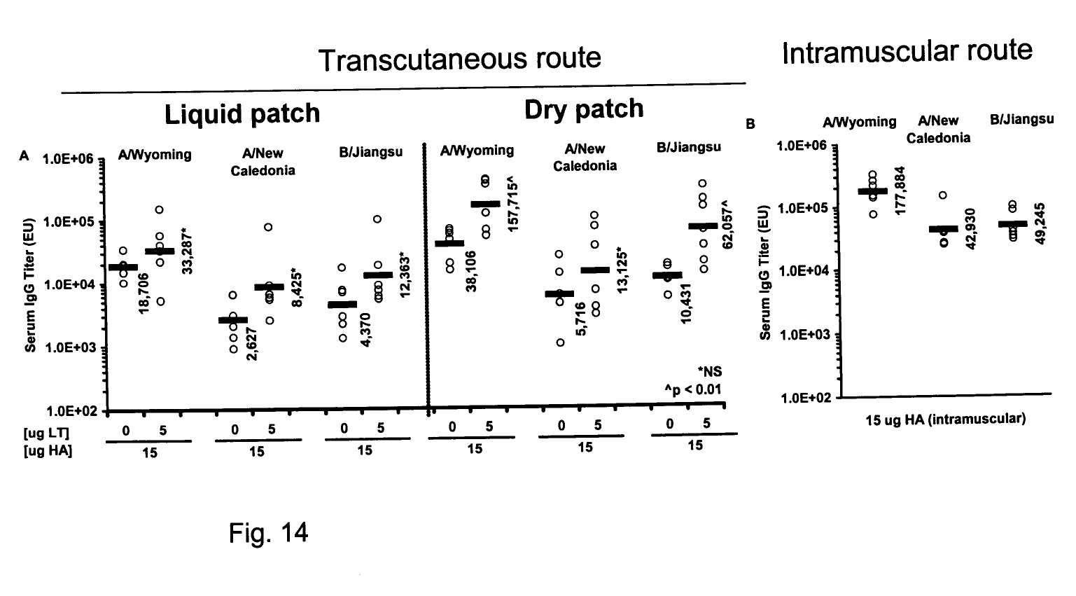

Results. As depicted in FIG. 14, Serum IgG antibody titers against A/Wyoming, A/New Caldeonia and B/Jiangsu strains in the absence or presence of the adjuvant (LT) in guinea pigs using the liquid patch or dry patch was measured. In guinea pigs treated with the ViaDerm device and applied with a dry patch, the IgG antibody titers against the strains were significantly higher than those obtained from guinea pigs treated with the ViaDerm device and applied with a liquid patch. Levels in both sets of guinea pigs being applied with either the dry or liquid patch still obtained immunologically effective titer levels of IgG in serum.

### Example 5

### Immune Response to Flu Vaccine Delivered on Skin Pretreated with ViaDerm in Hairy Guinea Pigs

Materials. 50 female Hartley guinea pigs, 300-400 g, >7 weeks old (Charles River) and 50 female Hairless guinea pigs, 300-400 g, >7 weeks old (Charles River).

Inactivated influenzae vaccine: A/Wyoming/03/2003 (H3N2), lot#1028825-0012, 284 μgHA/ml; A/New Caledonia/20/99 (H1N1), lot#1028827-0016, 237 μgHA/ml; B/Jingsu/10/2003, lot#1028826-0009, 542 μgHA/ml.

BLT: Berna, 2.16 mg/ml

One-layer rayon square patch 1 cm2

1 cm2 rayon patch, formulated in dried sucrose containing 15 μg HA (5 μg per strain) alone or with LT (5 and 15 μg).

ViaDerm: Electrodies at a depth at 100 μm, cylinder shape.

Tegaderm 1624W: 3M, NDC 8333-1624-05, 6 cm×7 cm size.

Adhesive tape: 3M

10% glycerol in saline.

Immunization. Before immunization and shaving, guinea pigs were sedated with ketamine and xylazine by standard procedure. All animals were bolus intramuscular injected with 0.5 μg HA (0.17 μg HA each strain) in 100 μl 1×DPBS on study day 1.

Pretreatment. Hairy guinea pigs were shaved on the abdomen immediately before patch application on study day 28. The immunization site was marked with a permanent marker and the shaven skin will be pretreated as follows:

Groups 1-3 and 11-13 were pretreated by hydration of the skin with 10% glycerol/saline without rubbing the site. Baseline tewl measurements will be determined before skin hydration.

Groups 4-9 and 14-19 were pretreated with 4-applications with the Viaderm 100 μm device applied within a 1 cm² area on dry skin. Tewl measurements were determined right following pretreatment. The skin was hydrated with 10% glycerol/saline immediately before patch application.

Patch application. To insure proper patch adherence, patches were covered with a modified Tegaderm overlay. Animals were wrapped with adhesive tape. Patches will be applied for 18-24 hr, removed, and the skin rinsed with warm water.

Groups were dosed as follows:

Groups 1, 7, 11 and 17 were immunized using dry sucrose patches (1 cm²) containing 15 μg HA;

Groups 2, 8, 12 and 18 were immunized using dry sucrose patches (1 cm²) containing 15 μg HA+5 μg LT;

Groups 3, 9, 13 and 19 were immunized using dry sucrose patches (1 cm²) containing 15 μg HA+15 μg LT;

Groups 4 and 14 were immunized using a rayon 1 cm2 patch with 15 μg HA in ˜15 μl.

Groups 5 and 15 were immunized using a rayon 1 cm2 patch with 15 μg HA+5 μg LT in ˜50 μl.

Groups 6 and 16 were immunized using a rayon 1 cm2 patch with 15 μg HA+15 μg LT in ˜50 μl.

Groups 10 and 20 were boosted by intramuscular (IM) injection with 15 μg HA on study day 22.

Serum collection. Post immune (day 27 and 48) blood samples were collected from the orbital plexus using standard methods. Serum was collected by centrifugation of whole blood and the cell free serum transferred to a labeled tube and stored frozen at −20° C.

ELISA: Sera was evaluated for total IgG titers to A/Wyoming, A/New Caledonia, B/Jiangsu and LT using an ELISA method. Antibody titers were reported as ELISA Units (EU), which is the serum dilution equal to 1OD at 405 nm. Student T test was used.

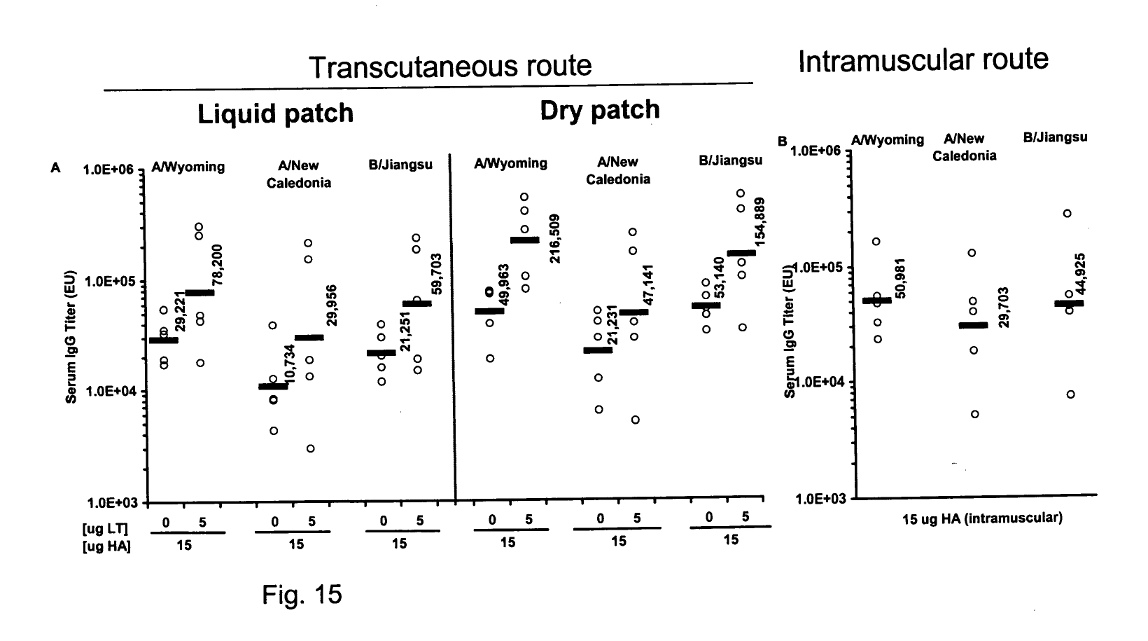

Results. As depicted in FIG. 15, serum IgG antibody titers against A/Wyoming, A/New Caldeonia and B/Jiangsu strains in guinea pigs using the liquid patch or dry patch or hydration alone was measured. In guinea pigs treated with the ViaDerm device and applied with a dry patch, the IgG antibody titers against the strains were significantly higher than those obtained from guinea pigs treated with the ViaDerm device and applied with a liquid patch. Levels in both sets of guinea pigs being applied with either the dry or liquid patch still obtained higher IgG titers than guinea pigs receiving a dry formulation of vaccine with hydration alone. Levels in both sets of guinea pigs being applied with either the dry or liquid patch still obtained immunologically effective titer levels of IgG in serum.

### Example 6

### Influenza Vaccine Adjuvanted with Berna LT and iLT in Guinea Pig

Materials. 66 female Hartley guinea pigs, 300-400 g, >7 weeks old (Charles River).

TIV: A/Wyoming/03/2003 (H3N2), lot#1028825-0012, 284 ugHA/ml; A/New Caledonia/20/99 (H1N1), lot#1028827-0016, 237 ugHA/ml; B/Jingsu/10/2003, lot#1028826-0009, 542 ugHA/ml.

Berna LT: lot #0007-008-08

iLT: lot #0408-002 (tox lot)

1 cm2 rayon patch, dry formulated containing 15 μg HA (5 μg per strain) alone or with BLT or iLT (5 and 15 μg)

ECG prep pad: Marquette Medical Systems, #9386-001

ViaDerm: Electrode Depth at <50 um, conic shape

Tegaderm 1624W: 3M, NDC 8333-1624-05, 6 cm×7 cm size

Adhesive tape: 3M

10% glycerol in salinei

Immunization. Im prime on Day 1: All animals were immunized by bolus intramuscular (IM) injection of 0.1 μg HA (0.033 μg each strain) in 100 ul PBS into the right thigh muscle.

Pretreatment. Guinea pigs were shaved on the abdomen immediately before patch application on study day 28. The immunization site was marked with a permanent marker and the shaven skin was pretreated as follows:

Groups 1-3 and 7-8 were pretreated with ECG prep pad 2× on dry skin. The skin was hydrated with 10% glycerol/saline immediately before patch application.

Groups 4-6 and 9-10 were pretreated with 4-applications with the Viaderm, <50 um device applied within a 1 cm² area on dry skin. Tewl measurements were determined right after pretreatment. The skin was hydrated with 10% glycerol/saline immediately before patch application.

Dosing. A 1 cm² dry formulated patch will be applied to immediately following treatment and hydration. Groups were dosed as follows:

Groups 1 and 4 received patches loaded with 15 μg HA alone.

Groups 2, 5, 7 and 9 received patches loaded with 15 μg HA and 5 μg LT.

Groups 3, 6, 8 and 10 received patches loaded with 15 μg HA and 15 μg LT.

Group 11 were boosted by intramuscular (IM) injection with 15 μg HA on study day 28.

Patch application. To insure proper patch adherence, patches will be covered with a modified Tegaderm overlay. Animals will be wrapped with adhesive tape. Patches will be applied for 18-24 hr, removed, and the skin rinsed with warm water.

Serum collection. Post immune (day 27 and 42) blood samples were collected from the orbital plexus using standard methods. Serum was collected by centrifugation of whole blood and the cell free serum transferred to a labeled tube and stored frozen at −20° C.

ELISA: Sera was evaluated for total IgG titers to A/Wyoming, A/New Caledonia, B/Jiangsu and LT using an ELISA method. Antibody titers were reported as ELISA Units (EU), which is the serum dilution equal to 1OD at 405 nm. Student T test was used.

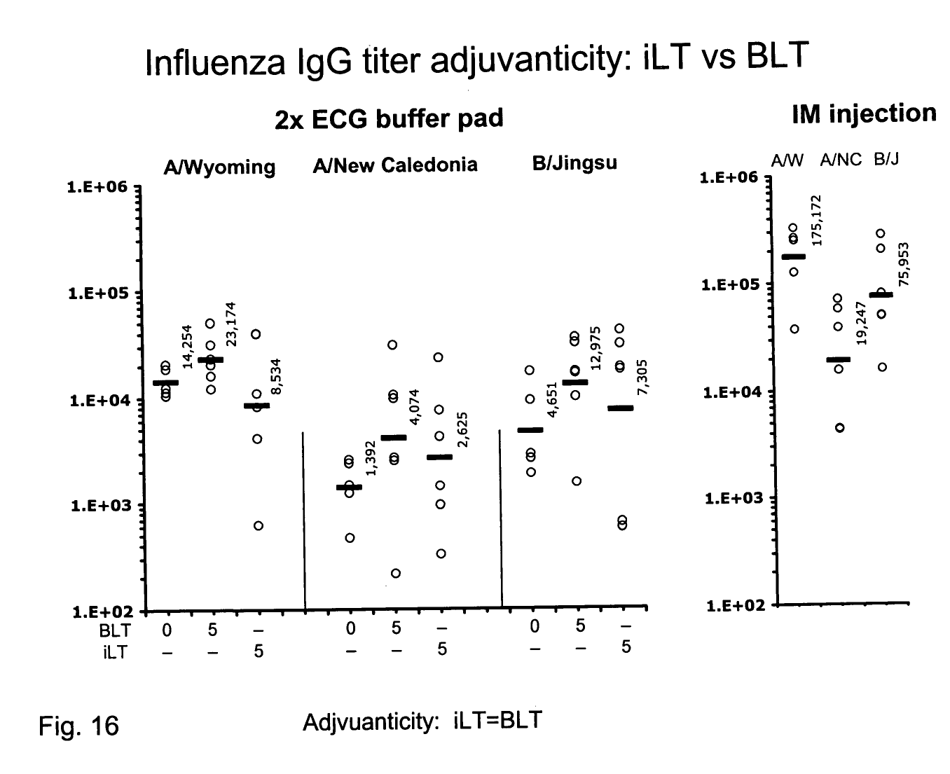

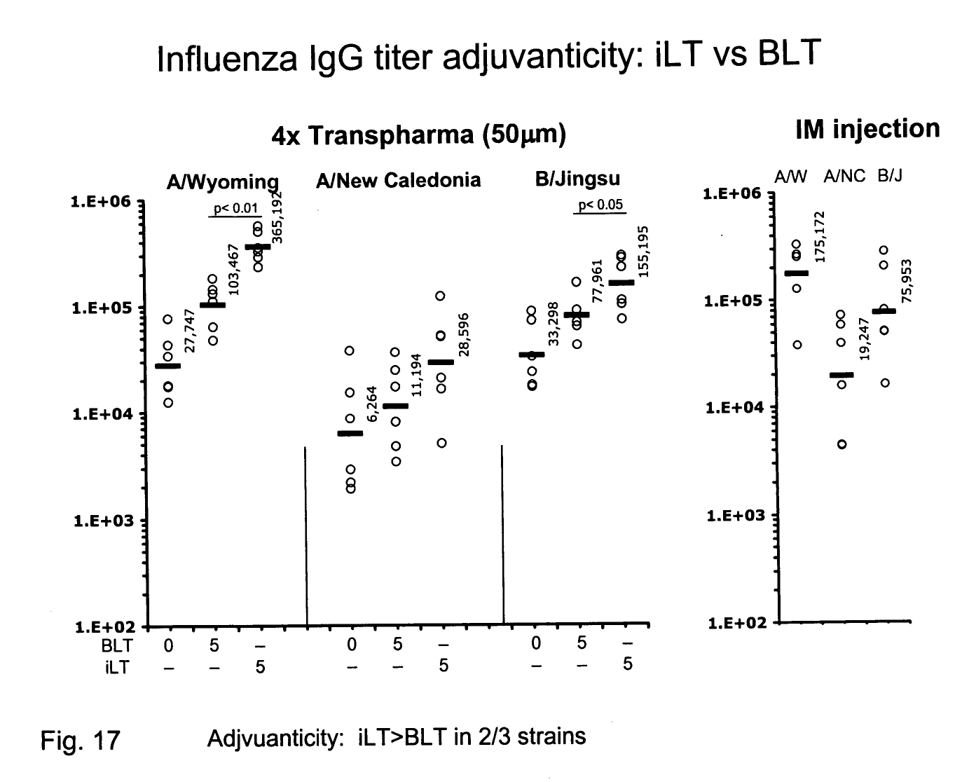

Results. As depicted in FIGS. 16 and 17, serum IgG antibody titers against A/Wyoming, A/New Caldeonia and B/Jiangsu strains in guinea pigs using a ECG pad or ViaDerm was measured. As shown in FIG. 17, in guinea pigs treated with the ViaDerm device and applied with a dry patch, the IgG antibody titers against the strains were significantly higher than those obtained from guinea pigs treated with the ECG pad and substantially similar to levels obtained from IM injection. Levels in guinea pigs receiving adjuvant with the ViaDerm device were higher than guinea pigs with no adjuvant. The IgG antibody titer levels in guinea pigs receiving the iLT adjuvant were higher in two out of the three strains than those receiving BLT.

It will be appreciated by persons skilled in the art that the present invention is not limited by what has been particularly shown and described herein above. Rather the scope of the invention is defined by the claims that follow.

1. A method for transdermally delivering a vaccine to at least one subject comprising:
applying a plurality of electrodes to the skin of said at least one subject;
generating a plurality of micro-channels through the stratum corneum of the skin by applying an electrical current through the skin; and
delivering said vaccine to the skin in the vicinity of said micro-channels.

2. The method of claim 1, wherein said electrical current is an alternating current at radio frequency.

3. The method of claim 2, wherein said the frequency of said alternating current is above about 100 Hz.

4. The method of claim 2, wherein said frequency of said alternating current is between about 1 kHz and about 300 kHz.

5. The method of claim 1, wherein plurality of electrodes arranged in an array about 80 μm in diameter and about 100 μm in length.

6. The method of claim 1, wherein the depth of said micro-channels is substantially equal to the length of the electrode.

7. The method of claim 1, wherein said electrical current further comprises a starting amplitude of about 140 V to about 350 V.

8. The method of claim 1, wherein said electrical current further comprises a starting amplitude of about 290 V.

9. The method of claim 1, wherein the depth of said micro-channels is related to the length of the electrodes and the potential difference applied between said electrodes.

10. The method of claim 1, wherein said electrical current further comprises a burst length of about 700 μsec to about 13,000 μsec.

11. The method of claim 1, wherein said electrical current further comprises a burst length of about 9000 μsec.

12. The method of claim 1, wherein the depth of said micro-channels is related to the length of the electrodes, the potential difference applied between said electrodes, and the time of application to said potential difference.

13. The method of claim 1, wherein said micro-channels are at a density of between about 100 micro-channels/cm to about 450 micro-channels/cm.

14. The method of claim 1, wherein said vaccine is delivered in a therapeutically effective amount.

15. The method of claim 14, wherein said therapeutically effective amount further comprise between about 0.1 μg to about 10 mg of antigen.

16. The method of claim 14, wherein IgA, IgM, and IgG are produced in response to the delivery of said therapeutically effective amount of said vaccine.

17. The method of claim 1, further comprising the initiation of a non-specific immune response in response to the generation of micro-channels.

18. The method of claim 17, wherein said non-specific immune response enhances vaccine delivery.

19. The method of claim 1, wherein said vaccine is delivered via a patch.

20. The method of claim 1, wherein said electrical current is applied to at least about 1 cm² of the skin of said at least one subject.

21. The method of claim 1, wherein the skin of said at least one subject is hydrated with a hydration agent following application of said electrical current and prior delivery of said vaccine.

22. The method of claim 19, wherein said hydration agent comprises 10% glycerol/saline.

23. A device for transdermally delivering a therapeutically effective amount of a vaccine to at least one subject comprising:
an electrode cartridge comprising a plurality of electrodes;
a control unit which is adapted to apply electrical energy at radio frequency to said plurality of electrodes when said plurality of electrodes are in the vicinity the skin of said subject wherein the application of said electrical energy generates a plurality of micro-channels through at least the stratum corneum of the skin; and
a patch comprising a vaccine comprising at least one antigen that is applied to the skin in the vicinity of said electrode cartridge following the application of electrical current.

24. The device of claim 23, wherein said plurality of electrodes further comprise arrays about 80 μm in diameter and about 100 μm in length.

25. The device of claim 21, wherein application of said electrical energy produces at least about 100 micro-channels/cm² to about 450 micro-channel/cm².
